(19) United States
(12) Patent Application Publication
(10) Pub. No.: US 2024/0000142 A1
(43) Pub. Date: Jan. 4, 2024

(54) AEROSOL GENERATING DEVICES FOR AQUEOUS CANNABINOID COMPOSITIONS

(71) Applicant: OMEGA LIFE SCIENCE LTD., Migdal Haemeq (IL)

(72) Inventor: Miron HAZANI, Haifa (IL)

(21) Appl. No.: 18/037,754

(22) PCT Filed: Nov. 22, 2021
(86) PCT No.: PCT/IL2021/051388

§ 371 Date: May 18, 2023

Related U.S. Application Data
(60) Provisional application No. 63/116,864, filed on Nov. 22, 2020.

(51) Int. Cl. A24F 40/42; A61M 11/04; A24F 40/10; A24F 40/46; A24F 40/48; A24B 15/167; A24F 40/44

(52) U.S. Cl. CPC A24F 40/42; A61M 11/042; A24F 40/10; A24F 40/46; A24F 40/48; A24B 15/167; A24F 40/44

(57) ABSTRACT

The present disclosure generally relates to the field of aerosol generation devices, and more particularly to vaporizers configured to generation of aerosols from aqueous formulations of cannabis products or nicotine products.

### FIELD OF THE INVENTION

The present disclosure generally relates to the field of aerosol generation devices, and more particularly to vaporizers configured to generation of aerosols from aqueous formulations of cannabis products or nicotine products.

### BACKGROUND OF THE INVENTION

Cannabis vaporizers typically function as condensation aerosol generators, which operate by vaporizing a liquid such as a raw or concentrated cannabis composition via heat applied by a heat source. Upon cooling, the vapor condenses to form an aerosol comprising droplets of liquid or particles which can be inhaled by a user through a mouthpiece.

The heated liquid in known vaporizers usually includes either (a) a composition or mixture of cannabinoids with organic solvents, having relatively low latent heat of vaporization, such as propylene glycol (PG) or vegetable glycerin (VG); (b) raw cannabis plant material; or (c) concentrated cannabis composition extracted from plant material. Said composition is typically referred to as “e-juice”.

An alternative to the above, which both (i) avoids the heating and chemical degradation and potentially harmful decomposition of organic solvents or structural plant material (e.g. leaves material); and (ii) enable the employment and inhalation of a less concentrated and more accurate and measured cannabis composition (cannabis concentrates are typically >80% cannabinoid, with the rest mostly organic solvents), would be the use of aqueous cannabinoid compositions.

However, cannabinoids are typically poorly soluble in water and water itself is difficult to vaporize because of its high latent heat value. Therefore, the field of aqueous cannabis compositions for inhalation and devices specifically configured for the aerosolization thereof for personal inhalation is under-developed. So, currently, devices for vaporization of aqueous compositions in general and of aqueous cannabinoid compositions in general, are very rare.

WO 2020/194297 is directed to an electronic cigarette comprising: a cartridge having a first end and a second end, the cartridge comprising: an evaporation heater configured to generate heat and to evaporate a liquid from a surface thereof; a liquid drawing element; a liquid container; and an outlet; and an actuator having a first end and a second end, the actuator comprising a processing unit, wherein the first end of the actuator is connectable with the second end of the cartridge, wherein the electronic cigarette further comprises a first trigger configured to generate a first trigger activation signal, and a liquid deposition mechanism comprising the liquid drawing element and the liquid container, wherein the liquid drawing element is spaced apart from the evaporation heater in at least a first state of the electronic cigarette, and wherein the liquid deposition mechanism is configured to transfer a discrete volume of an aqueous formulation from the liquid drawing element to the evaporation heater in a second state of the electronic cigarette, wherein the liquid drawing element is in contact with the liquid container in both the first state of the electronic cigarette and the second state of the electronic cigarette, wherein the processing unit is configured to receive at least one operation signal and to control operations of at least one of the evaporation heater and the liquid deposition mechanism upon receiving the at least one operation signal, wherein the at least one operation signal comprises the first trigger activation signal.

WO 2021/191891 discloses A cannabinoid composition suitable for administration of a cannabinoid via inhalation, the cannabinoid composition comprises an aqueous solution comprising at least one cannabinoic acid or a salt thereof, wherein the aqueous solution has a pH of at least 8.5.

There is an unmet need for a vaporizer capable of generating cannabinoid containing aerosol from aqueous cannabinoid compositions.

### SUMMARY OF THE INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

The present invention generally relates to the field of aerosol generation devices, and more particularly to vaporizers configured to generation of aerosols from aqueous formulations of cannabis and/or nicotine products.

According to some embodiments, the present invention provides an aerosol generating device cartridge configured to provide a concentrated aqueous composition to a user, including a concentrated aqueous cannabinoid compositions and aqueous nicotine compositions. According to some embodiments, the aqueous compositions are nicotine compositions. According to some embodiments, the aqueous compositions are cannabinoid compositions. Specifically, cannabinoids are known to be water insoluble, so aqueous compositions of cannabinoids have limited cannabinoid concentrations. Also, aqueous compositions of nicotine are sometimes restricted to relatively low concentrations. Aqueous compositions are highly desired for inhalation purposes, since water is safe to use via inhalation. However, aqueous cannabinoid composition for inhalation suffer from limited cannabinoid concentration due to the poor solubility of cannabinoid in water. This is a limitation for users, who prefer the sensation of concentrated cannabinoid compositions. Thus, nowadays concentrated cannabinoid compositions are provided in a non-aqueous form (e.g., a concentrate or in hydrophobic organic oily solvents).

According to some embodiments, there is provided an aerosol generating device cartridge having a proximal end and a distal end, wherein the cartridge comprises: a barrel having a proximal open face and a distal face, wherein the distal face is facing and located proximally to the distal cartridge end, wherein the barrel comprises an aqueous composition comprising at least one active compound suitable for inhalation; a concentrating module having a distal open vaporizable face and a proximal open face, wherein the concentrating module distal open face is in contact with the barrel, wherein the concentrating module is surrounded by at least one wall and is containing a packing material, wherein the packing material has higher affinity to the vaporizable active compound than its affinity to water; an outlet at the cartridge proximal end; and at least one evaporative heater configured to generate heat and to evaporate water from a surface thereof, wherein the evaporative heater is located proximally to the concentrating module proximal open face. According to some embodiments, the active compound is selected from nicotine and at least one cannabinoid. According to some embodiments, the active compound is nicotine. According to some embodiments, the active compound is at least one cannabinoid. According to some embodiments, the aqueous composition is an aqueous cannabinoid composition. According to some embodiments, the aqueous composition is an aqueous nicotine composition.

According to some embodiments, there is provided an aerosol generating device cartridge having a proximal end and a distal end, wherein the cartridge comprises: a barrel having a proximal open face and a distal face, wherein the distal face is facing and located proximally to the distal cartridge end, wherein the barrel comprises an aqueous composition comprising at least one vaporizable compound selected from: a cannabinoid, nicotine, or both; a concentrating module having a distal open face and a proximal open face, wherein the concentrating module distal open face is in contact with the barrel, wherein the concentrating module is surrounded by at least one wall and is containing a packing material, wherein the packing material has higher affinity to the at least one vaporizable compound than its affinity to water; an outlet at the cartridge proximal end; and at least one evaporative heater configured to generate heat and to evaporate water from a surface thereof, wherein the evaporative heater is located proximally to the concentrating module proximal open face.

According to some embodiments, there is provided an aerosol generating device cartridge having a proximal end and a distal end, wherein the cartridge comprises: a barrel having a proximal open face and a distal face, wherein the distal face is facing and located proximally to the distal cartridge end, wherein the barrel comprises an aqueous composition comprising at least one vaporizable compound selected from: a cannabinoid, nicotine, or both; a concentrating module having a distal open face and a proximal open face, wherein the concentrating module distal open face is in contact with the barrel, wherein the concentrating module is surrounded by at least one wall and is containing a chromatographic packing material; an outlet at the cartridge proximal end; and at least one evaporative heater configured to generate heat and to evaporate concentrated vaporizable compounds and water from a surface thereof, wherein the evaporative heater is located proximally to the concentrating module proximal open face.

According to some embodiments, the is provided an aerosol generating device cartridge having a proximal end and a distal end, wherein the cartridge comprises: a barrel having a proximal open face and a distal face, wherein the distal face is facing and located proximally to the distal cartridge end, wherein the barrel comprises an aqueous cannabinoid composition comprising at least one cannabinoid; a concentrating module having a distal open face and a proximal open face, wherein the concentrating module distal open face is in contact with the barrel, wherein the concentrating module is surrounded by at least one wall and is containing a packing material, wherein the packing material has higher affinity to the at least one cannabinoid than its affinity to water; an outlet at the cartridge proximal end; and at least one evaporative heater configured to generate heat and to evaporate water from a surface thereof, wherein the evaporative heater is located proximally to the concentrating module proximal open face.

According to some embodiments, the barrel is elongated. According to some embodiments, the barrel is structured as hollow and surrounded by at least one wall, thereby forming at internal cavity, wherein the aqueous composition resides within the internal cavity. According to some embodiments, the barrel proximal open face is facing the proximal cartridge face. According to some embodiments, the concentrating module distal open face is Facing distal cartridge end. According to some embodiments, the concentrating module proximal open face, is facing the cartridge proximal end. According to some embodiments, the evaporative heater is located proximally to the outlet.

According to some embodiments, the aerosol generating device further comprises a drain chamber. According to some embodiments, the drain chamber is located in proximity to the concentrating module. According to some embodiments, the drain chamber is located in proximity to the barrel. According to some embodiments, the drain chamber is located in proximity to the concentrating module and to the barrel. According to some embodiments, the drain chamber is contacting the concentrating module. According to some embodiments, the drain chamber is contacting the barrel. According to some embodiments, the drain chamber is contacting the concentrating module and to the barrel. According to some embodiments, the drain chamber has a shared wall with the concentrating module. According to some embodiments, the drain chamber has a shared wall the barrel. According to some embodiments, the drain chamber has a shared wall with each one of the contacting the concentrating module and to the barrel.

According to some embodiments, the aerosol generating device further comprises a drain chamber, wherein the at least one concentrating module wall comprises an opening, wherein the opening is allowing fluid communication between the drain chamber and the concentrating module.

According to some embodiments, the drain chamber is a substantially closed chamber, having at least one wall shared with the at least one concentrating module wall, wherein said shared wall comprises said opening, to allow the fluid communication.

According to some embodiments, upon a sufficient amount of the aqueous composition being transferred from the barrel to the concentrating module, the at least one vaporizable compound undergoes stronger retention to the packing material than the water thereby a dilute vaporizable compound composition is flowing to the drain chamber through the opening and a concentrated vaporizable compound composition is retained within the concentrating module.

According to some embodiments, upon a sufficient amount of the aqueous cannabinoid composition being transferred from the barrel to the concentrating module, the at least one cannabinoid undergoes stronger retention to the packing material than the water thereby a dilute cannabinoid composition is flowing to the drain chamber through the opening and a concentrated cannabinoid composition is retained within the concentrating module.

According to some embodiments, the concentration of the at least one vaporizable compound in the aqueous composition contained in the barrel is higher than the concentration of the at least one vaporizable compound in the dilute composition. According to some embodiments, the concentration of the at least one vaporizable compound in the aqueous composition contained in the barrel is lower than the concentration of the at least one vaporizable compound in the concentrated composition. According to some embodiments, the concentration of the at least one vaporizable compound in the aqueous composition contained in the barrel is higher than the concentration of the at least one vaporizable compound in the dilute composition and lower than the concentration of the at least one vaporizable compound in the concentrated composition.

According to some embodiments, the concentration of the at least one cannabinoid in the aqueous cannabinoid composition contained in the barrel is higher than the concentration of the at least one cannabinoid in the dilute cannabinoid composition. According to some embodiments, the concentration of the at least one cannabinoid in the aqueous cannabinoid composition contained in the barrel is lower than the concentration of the at least one cannabinoid in the concentrated cannabinoid composition. According to some embodiments, the concentration of the at least one cannabinoid in the aqueous cannabinoid composition contained in the barrel is higher than the concentration of the at least one cannabinoid in the dilute cannabinoid composition and lower than the concentration of the at least one cannabinoid in the concentrated cannabinoid composition.

According to some embodiments, upon a sufficient amount of the aqueous composition being transferred from the barrel to the concentrating module, the at least one vaporizable compound undergoes stronger retention to the packing material than the water, thereby a dilute composition is flowing to the drain chamber through the opening and a concentrated composition is retained within the concentrating module; wherein the concentration of the at least one vaporizable compound in the aqueous composition contained in the barrel is higher than the concentration of the at least one vaporizable compound in the dilute composition and lower than the concentration of the at least one vaporizable compound in the concentrated composition.

According to some embodiments, upon a sufficient amount of the aqueous cannabinoid composition being transferred from the barrel to the concentrating module, the at least one cannabinoid undergoes stronger retention to the packing material than the water, thereby a dilute cannabinoid composition is flowing to the drain chamber through the opening and a concentrated cannabinoid composition is retained within the concentrating module; wherein the concentration of the at least one cannabinoid in the aqueous cannabinoid composition contained in the barrel is higher than the concentration of the at least one cannabinoid in the dilute cannabinoid composition and lower than the concentration of the at least one cannabinoid in the concentrated cannabinoid composition.

According to some embodiments, the concentrating module has a total volume V_{c}, the packing material has a volume Vₛₚ and the remaining volume, V_{c}-Vₛₚ is free space, wherein upon a volume larger than V_{c}-Vₛₚ of the aqueous composition being transferred from the barrel to the concentrating module, the dilute composition is flowing to the drain chamber and the concentrated composition is retained within the concentrating module.

According to some embodiments, the retention factor of the at least one vaporizable compound upon elution of the composition through a column of the packing material is at least 2, at least 2.5, at least 3, at least 3.5, at least 4, at least 4.5, at least 5. Each possibility represents a separate embodiment of the invention. According to some embodiments, the retention factor of the at least one cannabinoid upon elution of the cannabinoid composition through a column of the packing material is at least 2.

According to some embodiments, the vaporizable compound is a cannabinoid. According to some embodiments, the packing material has higher affinity to the cannabinoid than its affinity to water. According to some embodiments, the vaporizable compound is a cannabinoid, wherein the packing material has higher affinity to the cannabinoid than its affinity to water.

According to some embodiments, the vaporizable compound is nicotine. According to some embodiments, the packing material has higher affinity to nicotine than its affinity to water. According to some embodiments, the vaporizable compound is nicotine, wherein the packing material has higher affinity to nicotine than its affinity to water.

According to some embodiments, the packing material is a chromatographic stationary phase packing material. According to some embodiments, the packing material is selected from the group consisting of: activated carbon, an ion exchange chromatography stationary phase packing material, a reversed-phase chromatography stationary phase packing material, a size exclusion chromatography stationary phase packing material and a combination thereof.

According to some embodiments, the chromatographic stationary phase packing material comprises silica, alumina, zirconia, titania, a cross linked polymer, a derivative or combination thereof. According to some embodiments, the chromatographic stationary phase packing material comprises a solid support material selected from silica, alumina, zirconia, titania, a cross linked polymer, derivatives and combination thereof.

According to some embodiments, the stationary phase packing material has pore size in the range of 60-4000 Angstrom.

According to some embodiments, the packing material is a reversed-phase chromatography packing material.

According to some embodiments, the packing material is represented by Formula I:

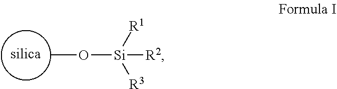

wherein
each one of R¹, R² and R³ is independently selected from the group consisting of a (C₁-C₃₀)-alkyl, a cycloalkyl, an aryl and a heteroaryl, each of which if optionally substituted with at least one substituent selected from the group consisting of halogen, nitro (NO₂), (C₁-C₃₀)-alkyl, C₂-C₃₀ alkenyl, C₂-C₃₀ alkynyl, cycloalkyl, aryl, heterocyclyl, heteroaryl, (C₁-C₃₀)-alkylaryl, (C₁-C₃₀)-alkylcycloalkyl, (C₁-C₃₀)-alkylheterocyclyl, (C₁-C₃₀)-alkylheteroaryl, haloalkyl, hydroxy (OH), (C₁-C₃₀)-alkyloxy, aryloxy, thio (SH), (C₁-C₃₀)-alkylthio, arylthio, —C(O)R, —C(O)OR, —X—(CH₂)ₜ-G, amino (NH₂), —NRR′, —C(O)NRR′, —S(O)OR, —S(O)₂OR, —S(O)NRR′ and —S(O)₂NRR′;
R and R′ are each independently H, (C₁-C₃₀)-alkyl, cycloalkyl or an aryl; and wherein each of said alkyl, cycloalkyl, heterocyclyl, aryl and heteroaryl is optionally substituted;
X is a bond, O, S or NH;
G is an acidic or basic moiety; and
each one of n and t is independently an integer in the range of 0 to 30;
and salts thereof.

According to some embodiments, each one of R¹ and R² is an unsubstituted (C₁-C₄)-alkyl and R³ is an unsubstituted (C₄-C₃₀)-alkyl.

According to some embodiments, at least one of R¹, R² and R³ is C₁₈H₃₇. According to some embodiments, at least one of R¹, R² and R³ is an unsubstituted (C₁-C₄)-alkyl.

According to some embodiments, each one of R¹ and R² is an unsubstituted (C₁-C₄)-alkyl and R³ is an unsubstituted (C₄-C₃₀)-alkyl. According to some embodiments, each one of R¹ and R² is CH₃ and R³ is an unsubstituted (C₈-C₁₈)-alkyl.

According to some embodiments, the packing material is a polymer-coated silica-based phase.

According to some embodiments, the packing material is a Porous Graphitic Carbon (PGC).

Porous Graphitic Carbon (PGC) is composed entirely of sheets of hexagonal arranged carbon atoms showing sp² hybridization. PGC has regular, homogeneous, flat surface and random, amorphous surface, which is associated with conventional silica-based stationary phases.

According to some embodiments, the packing material is a carbon-based chromatographic support material. According to some embodiments, the packing material comprises activated carbon. According to some embodiments, the packing material is activated carbon.

According to some embodiments, the packing material is an ion exchange chromatography packing material. According to some embodiments, the ion exchange chromatography packing material is selected from the group consisting of an anion exchange chromatography packing material and cation exchange chromatography packing material. According to some embodiments, the ion exchange chromatography packing material is a cation exchange chromatography packing material. According to some embodiments, the ion exchange chromatography packing material is an anion exchange chromatography packing material.

According to some embodiments, the ion exchange chromatography packing material comprises a matrix chemically bonded to an active group, wherein the matrix is selected from styrene-divinylbenzene copolymer gel, macromolecular styrene-divinylbenzene copolymer, macromolecular acrylic copolymer and acrylic gel and wherein the active group is selected from the group consisting of: tertiary amine, polyamine, N-methylglucamine free base, dimethylamino, quaternary ammonium functional group, benzyldimethyl(2-hydroxyethyl)ammonium functional group, dimethylethanolbenzyl ammonium, dimethylethanolamine, trimethylbenzylammonium dimethylethanolamine functional group and trimethylammonium.

According to some embodiments, the packing material is a size exclusion chromatography packing material.

According to some embodiments, the aqueous cannabinoid composition is selected from the group consisting of an aqueous cannabinoid solution, an aqueous cannabinoid emulsion and an aqueous cannabinoid suspension. Each possibility represents a separate embodiment of the invention. According to some embodiments, the aqueous cannabinoid composition is an aqueous cannabinoid solution.

According to some embodiments, the aqueous nicotine composition is an aqueous nicotine solution.

According to some embodiments, the cannabinoid is selected from the group consisting of cannabidiol, tetrahydrocannabinol, cannabidiolic acid, tetrahydrocannabinolic acid, salts thereof and combinations thereof According to some embodiments, the cannabinoid is a cannabinoid acid or a salt thereof. According to some embodiments, the cannabinoid acid is selected from the group consisting of tetrahydrocannabinolic acid, cannabidiolic acid and salts thereof.

According to some embodiments, the aqueous cannabinoid composition has a pH of at least 9. According to some embodiments, the aqueous cannabinoid composition has a pH of at least 10. According to some embodiments, the aqueous cannabinoid composition has a pH higher than 9. According to some embodiments, the aqueous cannabinoid composition has a pH higher than 10. According to some embodiments, the aqueous aerosol has a pH in the range of 5.5 to 7.5.

According to some embodiments, the aqueous composition comprises at least one vaporizable compound at a concentration of 0.5 to 10% w/w. According to some embodiments, the aqueous composition comprises at least one vaporizable compound at a concentration of 1 to 5% w/w.

According to some embodiments, the aqueous composition comprises at least one cannabinoid at a concentration of 0.5 to 10% w/w. According to some embodiments, the aqueous composition comprises at least one cannabinoid compound at a concentration of 0.2 to 5% w/w. According to some embodiments, the aqueous composition comprises at least one cannabinoid compound at a concentration of 1 to 5% w/w.

According to some embodiments, the aqueous composition comprises nicotine at a concentration of 0.5 to 10% w/w. According to some embodiments, the aqueous composition comprises nicotine at a concentration of 1 to 5% w/w. According to some embodiments, the aqueous composition comprises nicotine at a concentration of 0.2 to 2% w/w.

According to some embodiments, the aqueous cannabinoid composition comprises at least one cannabinoid at a concentration of at least 3% w/w. According to some embodiments, the aqueous cannabinoid composition comprises at least one cannabinoid at a concentration of at least 5% w/w. According to some embodiments, the concentration of the at least one cannabinoid compound in the aqueous cannabinoid composition is in the range of 2% to 20% w/w.

According to some embodiments, the aqueous composition comprises at least one cannabinoid at a concentration of no more than 2% w/w. According to some embodiments, the aqueous nicotine composition comprises nicotine at a concentration of no more than 2% w/w.

According to some embodiments, the concentration of the vaporizable compound in the concentrated composition in the concentrating module is in the range of 10% to 90% w/w. According to some embodiments, the concentration of the vaporizable compound in the concentrated composition is in the range of 10% to 90% w/w. According to some embodiments, the concentration of the vaporizable compound in the concentrated composition in the concentrating module is in the range of 30% to 90% w/w. According to some embodiments, the concentration of the vaporizable compound in the concentrated composition is in the range of 30% to 90% w/w. According to some embodiments, the concentration of the vaporizable compound in the concentrated composition in the concentrating module is in the range of 50% to 90% w/w. According to some embodiments, the concentration of the vaporizable compound in the concentrated composition is in the range of 50% to 90% w/w. According to some embodiments, the concentration of the vaporizable compound in the concentrated composition in the concentrating module is in the range of 60% to 90% w/w. According to some embodiments, the concentration of the vaporizable compound in the concentrated composition is in the range of 60% to 90% w/w. According to some embodiments, the concentration of the vaporizable compound in the concentrated composition in the concentrating module is in the range of 70% to 90% w/w. According to some embodiments, the concentration of the vaporizable compound in the concentrated composition is in the range of 70% to 90% w/w. According to some embodiments, the concentration of the vaporizable compound in the concentrated composition in the concentrating module is at least 40% w/w. According to some embodiments, the concentration of the vaporizable compound in the concentrated composition is at least 40% w/w. According to some embodiments, the concentration of the vaporizable compound in the concentrated composition in the concentrating module is at least 50% w/w. According to some embodiments, the concentration of the vaporizable compound in the concentrated composition is at least 50% w/w. According to some embodiments, the concentration of the vaporizable compound in the concentrated composition in the concentrating module is at least 60% w/w. According to some embodiments, the concentration of the vaporizable compound in the concentrated composition is at least 60% w/w. According to some embodiments, the concentration of the vaporizable compound in the concentrated composition in the concentrating module is at least 70% w/w. According to some embodiments, the concentration of the vaporizable compound in the concentrated composition is at least 80% w/w. According to some embodiments, the concentration of the vaporizable compound in the concentrated composition in the concentrating module is at least 70% w/w. According to some embodiments, the concentration of the vaporizable compound in the concentrated composition is at least 80% w/w. According to some embodiments, the concentration of the vaporizable compound in the aqueous composition contained in the barrel is in the range of 1% to 5% w/w and the concentration of the vaporizable compound in the concentrated composition in the range of 10% to 90% w/w.

According to some embodiments, the aqueous composition comprises both nicotine and at least one cannabinoid. According to some embodiments, the aqueous composition is an aqueous nicotine composition and an aqueous cannabinoid composition.

According to some embodiments, the aqueous composition further comprises ethanol. According to some embodiments, the aqueous cannabinoid composition further comprises ethanol.

According to some embodiments, the at least one cannabinoid is the sole active ingredient in the cannabinoid composition. According to some embodiments, the composition comprises cannabinoid(s) as the only active ingredient. According to some embodiments, the nicotine is the sole active ingredient in the nicotine composition. According to some embodiments, the composition comprises nicotine as the only active ingredient.

According to some embodiments, the aqueous composition is substantially devoid of organic solvents.

According to some embodiments, the aqueous composition comprises at least 70% w/w water. According to some embodiments, the aqueous composition comprises at least 50%, at least 60%, at least 70%, at least 75%, at least 80%, at least 85%, or at least 90% water w/w.

According to some embodiments, the cartridge further comprises a mouthpiece. According to some embodiments, the mouthpiece is located proximally to the outlet. According to some embodiments, the cartridge further comprised a mouthpiece located proximally to the outlet.

According to some embodiments, the evaporative heater is configured to generate heat and to evaporate the aqueous composition from the surface thereof to form an aqueous aerosol comprising the at least one vaporizable compound.

According to some embodiments, the evaporative heater is at least partially permeable to the aqueous formulation or wherein the cartridge comprises a plurality of evaporative heaters, wherein upon application of pressure of a fluid against the heater or the heaters, the fluid can pass therethrough.

According to some embodiments, the evaporative heater is rigid. According to some embodiments, the evaporative heater is made of metal. According to some embodiments, the evaporative heater has two flat sides, which remain flat when liquid is pressed there through. According to some embodiments, the evaporative heater has a top flat surface and a bottom flat surface, which do not deform when liquid is pressed there through or pressed against at least one of the top surface or the bottom surface.

According to some embodiments the heating element is coiled and is embedded within the concentrating unit

According to some embodiments, the evaporative heater is configured to provide 3-7 W, 4⁻⁶ W, 4.5-5.9 W, 4.8-5.6 W, 5.0-5.4 W or 5.1-5.3 W per every μl of liquid deposited thereon.

According to some embodiments the heater is configured to provide 30-110 Watts

According to some embodiments, the evaporative heater has a total resistance in the range of 0.150 to 1.5Ω.

According to some embodiments, the evaporative heater is configured to provide an energy output of at least 30 Watts.

According to some embodiments, the barrel comprises a liquid absorbing element. According to some embodiments, the barrel is housing a liquid absorbing element. According to some embodiments, the liquid absorbing element is extending through the barrel. According to some embodiments, the liquid absorbing element is elongated. According to some embodiments, the liquid absorbing element is in fluid communication with the concentrating module. According to some embodiments, the liquid absorbing element is in contact with the concentrating module. According to some embodiments, the liquid absorbing element is in contact with the concentrating module distal open face. According to some embodiments, the barrel comprises a liquid absorbing element, which is in contact with the concentrating module distal open face.

According to some embodiments, the barrel further comprises at least one container. According to some embodiments, the container is in fluid communication with the liquid absorbing element. According to some embodiments, the container is in contact with the liquid absorbing element. According to some embodiments, the container contains part of the aqueous composition. According to some embodiments, another part of the aqueous composition is absorbed within the liquid absorbing element. According to some embodiments, the barrel further comprises at least one container, which is in fluid communication with the liquid absorbing element and contains part of the aqueous composition, wherein another part of the aqueous composition is absorbed within the liquid absorbing element.

According to some embodiments, the liquid absorbing element comprises a sponge, a wick or both. According to some embodiments, the liquid absorbing element is a sponge, a wick or both. According to some embodiments, the liquid absorbing element is selected from the group consisting of: a sponge, a wick and a combination thereof. According to some embodiments, the liquid absorbing element is a sponge. According to some embodiments, the liquid absorbing element is a wick. According to some embodiments, the liquid absorbing element comprises a sponge. According to some embodiments, the liquid absorbing element comprises a wick.

According to some embodiments, there is provided an aerosol generating device comprising the cartridge as disclosed herein and an actuator. According to some embodiments, the actuator is reversibly connectable to the cartridge. According to some embodiments, the actuator is reversibly connectable to the cartridge at the cartridge distal end. According to some embodiments, the aerosol generating device further comprises controller, configured to control the operation of the evaporative heater. According to some embodiments, there is provided an aerosol generating device comprising the cartridge as disclosed herein and an actuator, wherein the actuator is reversibly connectable to the cartridge at the cartridge distal end, wherein the aerosol generating device further comprises controller, configured to control the operation of the evaporative heater.

According to some embodiments, there is provided an aerosol generating device comprising the cartridge of disclosed herein and an actuator. According to some embodiments, the actuator is reversibly connectable to the cartridge at the cartridge distal end. According to some embodiments, the aerosol generating device further comprises means of transferring at least a portion of the aqueous composition from the barrel to the concentrating module.

According to some embodiments, there is provided an aerosol generating device comprising the cartridge of disclosed herein and an actuator, wherein the actuator is reversibly connectable to the cartridge at the cartridge distal end, wherein the aerosol generating device further comprises means of transferring at least a portion of the aqueous composition from the barrel to the concentrating module.

According to some embodiments, the means of transferring at least a portion of the aqueous composition from the barrel to the concentrating module is selected from a liquid pump and a plunger assembly.

According to some embodiments, the means of transferring at least a portion of the aqueous composition from the barrel to the concentrating module comprises a plunger assembly.

According to some embodiments, the plunger assembly comprises a plunger actuator, a rod and a plunger head, wherein the rod has a proximal end and a distal end, wherein the distal end is connected to the solenoid actuator, and the proximal end is connected to the plunger head, wherein the plunger actuator is configured to dislocate the plunger head in the proximal direction from a first position to a second position, thereby to force at least a portion of the aqueous composition from the barrel to the concentrating module.

According to some embodiments, the solenoid actuator is configured to receive electric current and to generate axial movement of the solenoid plunger head upon receiving the electric current, wherein the axial movement is along an elongation of the cartridge.

According to some embodiments, upon dislocating to the second position, the plunger head is at least partially within the barrel.

According to some embodiments, upon connecting the actuator and the cartridge and upon dislocating the plunger head to said second position, it is within the barrel.

According to some embodiments, the plunger actuator, the rod and the plunger head are disposed within the actuator, wherein upon connecting the actuator and the cartridge and upon dislocating the plunger head to said second position, it is within the barrel.

According to some embodiments, the plunger actuator is disposed within the actuator and the rod and the plunger head are disposed within the cartridge.

According to some embodiments, the aerosol generating device further comprises a plunger seal sealingly movable through at least an interior portion of the barrel, wherein the portion comprises at the distal barrel face, wherein the plunger seal is in contact with the aqueous composition, thereby sealing and preventing leakage thereof through the distal barrel face.

According to some embodiments, the seal is made of a polymeric material. According to some embodiments, the seal is made of rubber.

According to some embodiments, the plunger actuator is configured to dislocate the plunger head in the proximal direction from a first position to a second position, wherein upon said dislocation the plunger head is pressing against and forcing the plunger seal in the proximal direction through the interior portion of the barrel, thereby forcing at least a portion of the aqueous composition from the barrel to the concentrating module.

According to some embodiments, the means of transferring at least a portion of the aqueous composition from the barrel to the concentrating module comprises a liquid pump. According to some embodiments, the liquid pump is configured to apply pressure on the aqueous composition in the proximal direction, thereby forcing the aqueous composition from the barrel into the concentrating module.

According to some embodiments, there is provided a method of producing an aqueous aerosol, the method comprising:
(a) providing an aqueous composition comprising an at least one vaporizable compound selected from: a cannabinoid, nicotine, or both;
(b) providing a cartridge comprising a concentrating module and a at least one evaporative heater, wherein the concentrating module having a distal open face and a proximal open face, wherein the evaporative heater is located proximally to the concentrating module proximal open face
(c) passing the aqueous composition through the concentrating module, from the distal concentrating module open face towards the evaporative heater, thereby concentrating the aqueous composition; and
(d) operating the at least one evaporative heater, thereby producing an aqueous aerosol.

According to some embodiments, the concentrating module is surrounded by at least one wall and is containing a packing material, wherein the packing material has higher affinity to the vaporizable compound than its affinity to water, wherein passing the aqueous cannabinoid composition through the concentrating module in step (c) entails concentrating the aqueous composition.

According to some embodiments, the aqueous aerosol is selected from an aqueous cannabinoid aerosol and an aqueous nicotine aerosol. According to some embodiments, the aqueous aerosol is an aqueous cannabinoid aerosol. According to some embodiments, the aqueous aerosol is an aqueous nicotine aerosol.

According to some embodiments, the aqueous composition comprising active compound is selected from the group consisting of an aqueous cannabinoid composition comprising at least one cannabinoid, an aqueous nicotine composition comprising nicotine, and a combination thereof. According to some embodiments, the aqueous composition is an aqueous cannabinoid composition comprising at least one cannabinoid. According to some embodiments, the aqueous composition is an aqueous nicotine composition comprising nicotine.

According to some embodiments, there is provided a method of producing an aqueous cannabinoid aerosol, the method comprising:
(a) providing an aqueous cannabinoid composition comprising at least one cannabinoid;
(b) providing a cartridge comprising a concentrating module and a at least one evaporative heater, wherein the concentrating module having a distal open face and a proximal open face, wherein the evaporative heater is located proximally to the concentrating module proximal open face
(c) passing the aqueous cannabinoid composition through the concentrating module, from the distal concentrating module open face towards the evaporative heater, thereby concentrating the aqueous cannabinoid composition; and
(d) operating the at least one evaporative heater, thereby producing an aqueous cannabinoid aerosol.

According to some embodiments, the concentrating module is surrounded by at least one wall and is containing a packing material, wherein the packing material has higher affinity to the at least one cannabinoid than its affinity to water, wherein passing the aqueous cannabinoid composition through the concentrating module in step (c) entails concentrating the aqueous cannabinoid composition.

According to some embodiments, there is provided a method of producing an aqueous nicotine aerosol, the method comprising:
(a) providing an aqueous nicotine composition comprising nicotine;
(b) providing a cartridge comprising a concentrating module and a at least one evaporative heater, wherein the concentrating module having a distal open face and a proximal open face, wherein the evaporative heater is located proximally to the concentrating module proximal open face;
(c) passing the aqueous nicotine composition through the concentrating module, from the distal concentrating module open face towards the evaporative heater, thereby concentrating the aqueous nicotine composition; and
(d) operating the at least one evaporative heater, thereby producing an aqueous nicotine aerosol.

According to some embodiments, the concentrating module is surrounded by at least one wall and is containing a packing material, wherein the packing material has higher affinity to nicotine than its affinity to water, wherein passing the aqueous nicotine composition through the concentrating module in step (c) entails concentrating the aqueous nicotine composition.

According to some embodiments, there is provided a method of producing an aqueous aerosol, the method comprising:
(a) providing an aqueous nicotine composition comprising at least one vaporizable compound selected from: a cannabinoid, nicotine, or both;
(b) providing a cartridge comprising a concentrating module and a at least one evaporative heater, wherein the concentrating module having a distal open face and a proximal open face, wherein the evaporative heater is located proximally to the concentrating module proximal open face;
(c) passing the aqueous composition through the concentrating module, from the distal concentrating module open face towards the evaporative heater, thereby concentrating the aqueous composition; and
(d) operating the at least one evaporative heater, thereby producing an aqueous aerosol.

According to some embodiments, the cartridge is the cartridge described herein.

According to some embodiments, step (c) comprises applying liquid pressure of the aqueous composition in the proximal direction.

According to some embodiments, the concentrating the aqueous composition in step (c) comprises separating the aqueous composition into concentrated composition and a dilute composition. According to some embodiments, upon said separation, the concentrated composition resides within the concentrating module. According to some embodiments, upon said separation, the dilute composition resides out of the concentrating module. According to some embodiments, upon said separation, the dilute composition resides out of the barrel. According to some embodiments, upon said separation, the dilute composition resides within the drain chamber. According to some embodiments, the concentration of the at least one vaporizable compound in the aqueous composition is higher than the concentration of the at least one vaporizable compound in the dilute composition and lower than the concentration of the at least one vaporizable compound in the concentrated composition. According to some embodiments, the concentration of the at least one vaporizable compound in the aqueous composition prior to the separation is higher than the concentration of the at least one vaporizable compound in the dilute composition and lower than the concentration of the at least one vaporizable compound in the concentrated composition. According to some embodiments, the concentration of the at least one cannabinoid in the aqueous composition is in the range of 1% to 10% w/w, wherein concentrating the aqueous composition in step (c) entails at least doubling said concentration.

According to some embodiments, the concentrating the aqueous cannabinoid composition in step (c) comprises separating the aqueous cannabinoid composition into concentrated cannabinoid composition and a dilute cannabinoid composition. According to some embodiments, upon said separation, the concentrated cannabinoid composition resides within the concentrating module. According to some embodiments, upon said separation, the dilute composition resides out of the concentrating module. According to some embodiments, upon said separation, the dilute composition resides out of the barrel. According to some embodiments, upon said separation, the dilute composition resides within the drain chamber. According to some embodiments, the concentration of the at least one cannabinoid in the aqueous cannabinoid composition is higher than the concentration of the at least one cannabinoid in the dilute cannabinoid composition and lower than the concentration of the at least one cannabinoid in the concentrated cannabinoid composition. According to some embodiments, the concentration of the at least one cannabinoid in the aqueous cannabinoid composition prior to the separation is higher than the concentration of the at least one cannabinoid in the dilute cannabinoid composition and lower than the concentration of the at least one cannabinoid in the concentrated cannabinoid composition. According to some embodiments, the concentration of the at least one cannabinoid in the aqueous cannabinoid composition is in the range of 1% to 10% w/w, wherein concentrating the aqueous cannabinoid composition in step (c) entails at least doubling said concentration.

According to some embodiments, step (c) further comprises flowing the dilute aqueous composition into the drain chamber.

According to some embodiments, the concentration of the at least one vaporizable compound in the concentrated composition of step (c) is in the range of 10% to 85% w/w.

According to some embodiments, there is provided an aqueous aerosol produced by the method of the present invention. According to some embodiments, the aqueous aerosol contains water and nicotine and/or at least one cannabinoid.

According to some embodiments, there is provided an aqueous cannabinoid aerosol produced by the method of the present invention.

According to some embodiments, there is provided an aqueous nicotine aerosol produced by the method of the present invention.

According to some embodiments, the aqueous aerosol has droplets having a mass median aerodynamic diameter (MMAD) in the range of 0.2-2 microns.

Other objects, features and advantages of the present invention will become clear from the following description, examples and drawings.

### BRIEF DESCRIPTION OF THE DRAWINGS

FIG. 1 constitutes a schematic view of a typical aerosol generating device cartridge. The cartridge of FIG. 1 contains cannabinoids (triangles).

FIG. 2 constitutes a schematic cross-sectional view of an aerosol generating device comprising a cartridge and an actuator, when connected, according to some embodiments.

FIG. 3 constitutes a schematic cross-sectional view of an aerosol generating device comprising a cartridge and an actuator, when separated, according to some embodiments.

FIG. 4 constitutes a schematic cross-sectional view of an aerosol generating device comprising a cartridge and an actuator, at stage A of initialization, according to some embodiments. The cartridge of FIG. 4 contains an aqueous composition comprising cannabinoid(s) and/or nicotine (triangles) and water (circles) and a concentrating module stationary comprising packing material (stars).

FIG. 5 constitutes a schematic cross-sectional view of an aerosol generating device comprising a cartridge and an actuator, at stage B of concentration, according to some embodiments. The cartridge of FIG. 5 contains an aqueous composition comprising cannabinoid(s) and/or nicotine (triangles) and water (circles) and a concentrating module stationary comprising packing material (stars).

FIG. 6 constitutes a schematic cross-sectional view of an aerosol generating device comprising a cartridge and an actuator, at stage C of concentration and drain, according to some embodiments. The cartridge of FIG. 6 contains an aqueous composition comprising cannabinoid(s) and/or nicotine (triangles) and water (circles) and a concentrating module stationary comprising packing material (stars).

FIG. 7 constitutes a schematic cross-sectional view of an aerosol generating device comprising a cartridge and an actuator, at stage D of concentration and drain, according to some embodiments. The cartridge of FIG. 7 contains an aqueous composition comprising cannabinoid(s) and/or nicotine (triangles) and water (circles) and a concentrating module stationary comprising packing material (stars).

FIG. 8 constitutes a schematic cross-sectional view of an aerosol generating device comprising a cartridge and an actuator, at stage E of aerosolization, according to some embodiments. The cartridge of FIG. 8 contains an aqueous composition comprising cannabinoid(s) and/or nicotine (triangles) and water (circles) and a concentrating module stationary comprising packing material (stars).

FIG. 9 constitutes a schematic cross-sectional view of an aerosol generating device comprising a cartridge and an actuator, when connected, according to some embodiments.

FIG. 10 constitutes a schematic cross-sectional view of an aerosol generating device comprising a cartridge and an actuator, when separated, according to some embodiments

FIG. 11 constitutes a schematic cross-sectional view of an aerosol generating device comprising a cartridge and an actuator, at stage A of initialization, according to some embodiments. The cartridge of FIG. 11 contains an aqueous composition comprising cannabinoid(s) and/or nicotine (triangles) and water (circles) and a concentrating module stationary comprising packing material (stars).

FIG. 12 constitutes a schematic cross-sectional view of an aerosol generating device comprising a cartridge and an actuator, at stage E of aerosolization, according to some embodiments. The cartridge of FIG. 12 contains an aqueous cannabinoid composition comprising cannabinoid(s) and/or nicotine (triangles) and water (circles) and a concentrating module stationary comprising packing material (stars).

### DETAILED DESCRIPTION

The present invention generally relates to the field of aerosol generation devices, and more particularly to vaporizers configured to generation of aerosols from aqueous formulations of cannabis products.

According to some embodiments, the present invention provides an aerosol generating device cartridge configured to provide a concentrated aqueous composition to a user via inhalation. Specifically, the aerosol generating device 200 and aerosol generating device cartridge 100 of the present invention are configured to receive a standard aqueous composition for inhalation, to concentrate said composition such that a concentrated aqueous composition is formed, and to vaporize said concentrated aqueous composition. The aerosol generating device cartridge 100 may be used for delivery of various aqueous compositions. Primarily compositions, which are referred herein are aqueous cannabinoid compositions and aqueous nicotine solutions. In all the cases, the concentration of the active material (e.g., cannabinoid(s), nicotine) is elevated in the process of using the cartridge 100, so that a concentrated composition is aerosolized. Aqueous cannabinoid compositions are specifically referred herein as cannabinoids are have typically poor aqueous solubility and cannot be provided as concentrated solutions in advance. Nicotine-water compositions are also referred herein as these are also typically provided in concentrations of up to 2%.

Cannabinoids are known to be water insoluble, so aqueous compositions of cannabinoids have limited cannabinoid concentrations. Aqueous compositions are highly desired for inhalation purposes, since water is safe to use via inhalation. However, aqueous cannabinoid compositions for inhalation suffer from limited cannabinoid concentration due to the poor solubility of cannabinoid in water. This is a limitation for users, who prefer the sensation of concentrated cannabinoid compositions. Thus, nowadays concentrated cannabinoid compositions are provided in a non-aqueous form (e.g., a concentrate or in hydrophobic organic oily solvents).

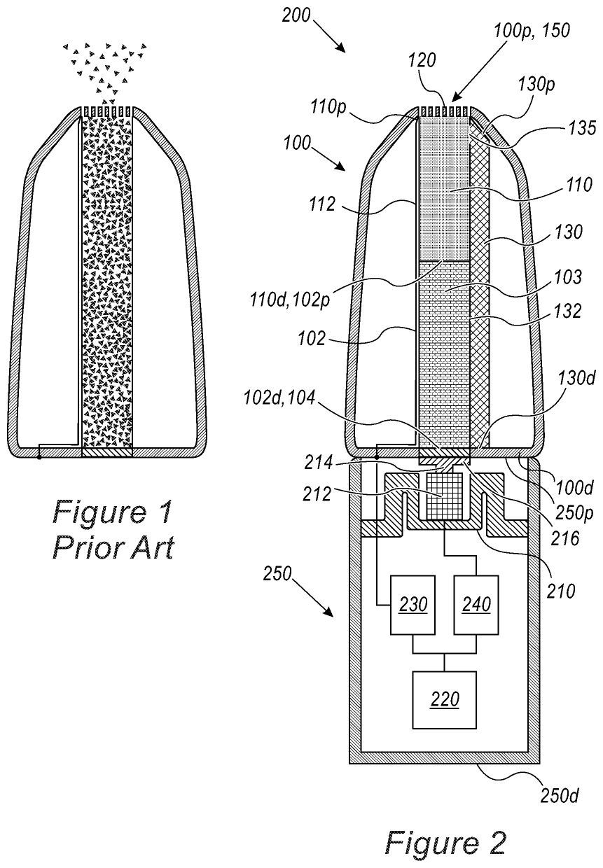

Reference is now made to FIG. 1, which presents an aerosol generating device cartridge used in the art. Such cartridge includes a concentrated cannabinoid composition, which consists essentially of cannabinoids (typically over 80% cannabinoids) in a viscous oil form. The cannabinoids are marked as triangles. In the cartridge of FIG. 1, the concentrated cannabinoid composition is in contact with a cartridge heater and upon activation of the heater vaporization of the cannabinoids occur to form an aerosol. Since the aerosol is made from a non-aqueous composition, is includes mainly cannabinoids and is devoid of water.

Reference is now made to FIGS. 2-12.

The present invention provides an aerosol generating device cartridge 100, which may be a part of an aerosol generating device (e.g. aerosol generating device 200), or a standalone cartridge to be connected to different aerosol generating device actuators, wherein the aerosol generating device cartridge 100 comprises a concentrating module 110 configured to receive a relatively dilute aqueous cannabinoid and/or nicotine compositions and to concentrate said dilute compositions to form a concentrated composition, which is ready for inhalation. According to some embodiments, the concentrating module 110 includes packing material, wherein the packing material has higher affinity to the at least one cannabinoid and/or nicotine than its affinity to water, such that the water component may pass therethrough, while cannabinoids and/or nicotine remain substantially within. This results in the concentrated cannabinoid and/or nicotine composition, which is ready for aerosolization and inhalation. According to some embodiments, the concentrating module 110 of the present invention is based on the principles or liquid chromatography, which is discussed in detail below.

According to some embodiments, the is provided an aerosol generating device cartridge 100 having an aerosol generating device cartridge proximal end 100p and an aerosol generating device cartridge distal end 100d, wherein the aerosol generating device cartridge 100 comprises: a barrel 102 having a barrel proximal open face 102p and a barrel distal open face 102d, wherein the barrel distal open face 102d is facing and located proximally to the aerosol generating device cartridge distal end 100d, wherein the barrel 102 comprises an aqueous composition comprising at least one cannabinoid and/or nicotine; a concentrating module 110 having a concentrating module distal open face 110d and a concentrating module proximal open face 110p, wherein the concentrating module distal open face 110d is in contact with the barrel 102, wherein the concentrating module 110 is surrounded by at least one wall 112 and is containing a packing material, wherein the packing material has higher affinity to the at least one cannabinoid and/or nicotine than its affinity to water; an outlet 150 at the aerosol generating device cartridge proximal end 100p; and at least one evaporative heater configured to generate heat and to evaporate water from a surface thereof, wherein the evaporative heater 120 is located proximally to the concentrating module proximal open face 110p.

As used herein the term “aerosol” refers to a suspension of solid or liquid particles in a gas. As used herein “aerosol” may be used generally to refer to a drug that has been vaporized, nebulized, or otherwise converted from a solid or liquid form to an inhalable form including suspended solid or liquid drug particles. According to some embodiments, the drug particles include THC particles.

The term “aerosol generating device” refer to a device configured to produce a vapor or aerosol from a liquid or solid composition. aerosol generating devices are typically used to deliver a solid or liquid (including semi liquid) composition to a subject in need thereof in a inhalable form (i.e. in a substantially gaseous form). Aerosol generating devices include nebulizers and inhalers, which typically produce aerosols by application of mechanical force on the compositions (e.g., by gas flow or vacuum), and to vaporizers and electronic cigarettes, which typically heating unit(s) and produce aerosols by vaporizing the composition. In both instances, the composition is delivered through an outlet, wherein in the latter instances (i.e., vaporizers and electronic cigarettes) the vapor is usually at least partially being condensed to form droplets of the composition, through the delivery.

The present aerosol generating devices include heating units, and are typically conventionally referred to as vaporizers. Specifically, the typical convention is that aerosol generating devices for aerosolizing nicotine/tobacco compositions are called electronic cigarettes, whereas devices for aerosolizing cannabinoid/cannabis compositions are called vaporizer or vaping devices.

The term “barrel” as used herein is to be defined as known for syringes, specifically, this is a hollow, typically elongated, and typically double open-ended element, which is configured to contain a liquid, while a solid element can be pushed therethrough thereby pushing the liquid in the same direction and out of one of the open ends, according to some embodiments.

According to some embodiments, the barrel 102 is elongated. According to some embodiments, the barrel 102 is structured as hollow and surrounded by at least one wall 112, thereby forming a barrel internal cavity 103, wherein the aqueous cannabinoid and/or nicotine composition resides within the barrel internal cavity 103. According to some embodiments, the barrel 102 is in the form of a cylinder. According to some embodiments, the barrel 102 is cylindrical. According to some embodiments, the barrel 102 is cuboid.

According to some embodiments, the barrel proximal open face 102p is facing the aerosol generating device cartridge proximal end 100p. According to some embodiments, the concentrating module distal open face 110d is facing aerosol generating device cartridge distal end 100d. According to some embodiments, the concentrating module proximal open face 110p, is facing the aerosol generating device cartridge proximal end 100p.

According to some embodiments, the evaporative heater 120 is located proximally to the outlet 150.

According to some embodiments, the aerosol generating device cartridge 100 further comprises a drain chamber 130. According to some embodiments, the drain chamber 130 is located in proximity to the concentrating module 110. According to some embodiments, the drain chamber 130 is located in proximity to the barrel 102. According to some embodiments, the drain chamber 130 is located in proximity to the concentrating module 110 and to the barrel 102. According to some embodiments, the drain chamber 130 is contacting the concentrating module 110. According to some embodiments, the drain chamber 130 is contacting the barrel 102. According to some embodiments, the drain chamber 130 is contacting the concentrating module 110 and to the barrel 102. According to some embodiments, the drain chamber 130 has a shared wall 132 with the concentrating module 110. According to some embodiments, the drain chamber 130 has a shared wall 132 the barrel 102. According to some embodiments, the drain chamber 130 has a shared wall 132 with each one of the contacting the concentrating module 110 and to the barrel 102.

It is to be understood that the figures are illustrative and do not necessarily reflect an actual scale. Specifically, in some of the figures the concentrating module 110 is about the same size as the barrel 102, although typically, according to some embodiments, the length of the barrel 102 (the distance between the barrel proximal open face 102p and the barrel distal open face 102d is higher than the length of the concentrating module 110 (the distance between concentrating module proximal open face 110p and concentrating module distal open face 110d). According to some embodiments, the length of the barrel 102 is at least 2, at least 3, at least 4, at least 5, at least 7.5, at least 10, at least 15, at least 20 or at least 25 higher than the length of the concentrating module 110. Each possibility represents a separate embodiment of the invention.

According to some embodiments, the drain chamber 130 is structured to contain liquids. According to some embodiments, the drain chamber 130 is surrounded by a wall and has a drain chamber proximal closed end 130p and a drain chamber distal closed end 130d. According to some embodiments, the drain chamber proximal closed end 130p is located in proximity to the concentrating module proximal open face 110p. According to some embodiments, the drain chamber distal closed end 130d is located in proximity to the barrel distal open face 102d. According to some embodiments, the drain chamber proximal closed end 130p is located in proximity to the concentrating module proximal open face 110p and the drain chamber distal closed end 130d is located in proximity to the barrel distal open face 102d.

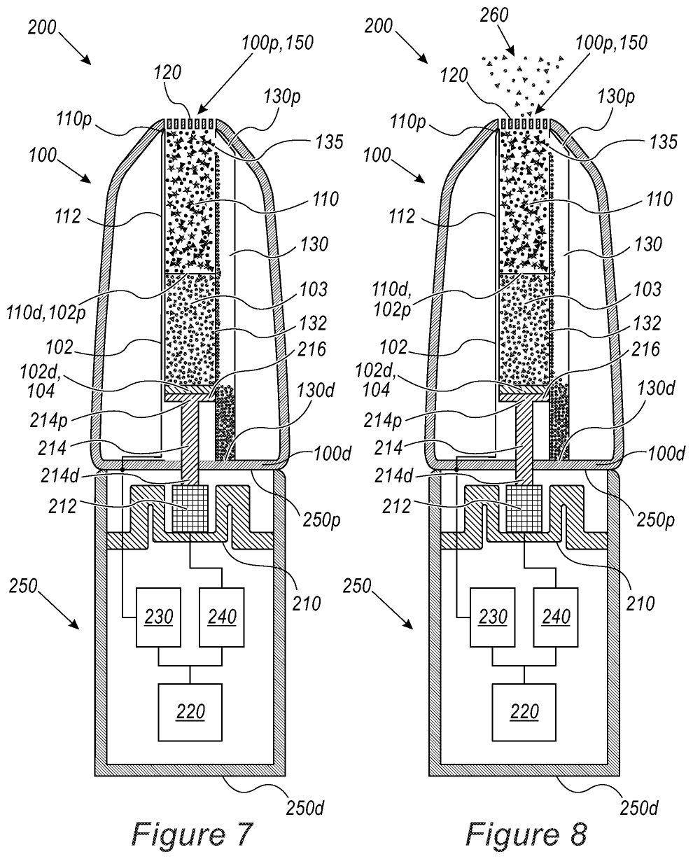

According to some embodiments, the at least one concentrating module wall 112 comprises an opening 135. According to some embodiments, the opening 135 is connecting between the concentrating module 110 and the drain chamber 130. According to some embodiments, the opening 135 is allowing fluid communication between the drain chamber 130 and the concentrating module 110. Specifically, as detailed below, and can be appreciated from FIG. 6 and FIG. 7, the concentrating module 110 is configured to separate the aqueous composition to a dilute composition comprising small amounts of the at least one cannabinoid and/or nicotine, and to a concentrated composition, which is aerosolized and inhaled, according to some embodiments. The dilute composition is to pass through the opening 135 to the drain chamber 130, when it is pooled or stocked until it is drained out of the aerosol generating device cartridge 100 or until the consumption of the vaporizable compounds (cannabinoids and/or nicotine) within the aerosol generating device cartridge 100.

According to some embodiments, the aerosol generating device cartridge 100 further comprises a drain chamber 130, wherein the at least one concentrating module wall 112 comprises an opening 135, wherein the opening 135 is allowing fluid communication between the drain chamber 130 and concentrating module 110.

According to some embodiments, the drain chamber 130 is a substantially closed chamber, having at least one wall 132 shared with the at least one concentrating module wall 112, wherein said shared wall 132 comprises said opening 135.

According to some embodiments, the drain chamber 130 is a substantially closed chamber, having at least one wall 132 shared with the at least one concentrating module wall 112, wherein said shared wall 132 comprises said opening 135, to allow the fluid communication.

According to some embodiments, upon a sufficient amount of the aqueous composition being transferred from the barrel 102 to the concentrating module 110, the at least one vaporizable compound undergoes stronger retention to the packing material than the water. According to some embodiments, upon a sufficient amount of the aqueous composition being transferred from the barrel 102 to the concentrating module 110, the at least one vaporizable compound undergoes stronger retention to the packing material than the water thereby a dilute cannabinoid and/or nicotine composition is flowing to the drain chamber 130 through the opening 135.

According to some embodiments, upon a sufficient amount of the aqueous composition being transferred from the barrel 102 to the concentrating module 110, the at least one vaporizable compound undergoes stronger retention to the packing material than the water thereby a dilute cannabinoid and/or nicotine composition is flowing to the drain chamber 130 through the opening 135 and a concentrated cannabinoid and/or nicotine composition is retained within the concentrating module 110.

According to some embodiments, the concentration of the at least one vaporizable compound in the aqueous cannabinoid composition contained in the barrel 102 is higher than the concentration of the at least one vaporizable compound in the dilute vaporizable compound composition. According to some embodiments, the concentration of the at least one vaporizable compound in the aqueous vaporizable compound composition contained in the barrel 102 is lower than the concentration of the at least one vaporizable compound in the concentrated vaporizable compound composition. According to some embodiments, the concentration of the at least one vaporizable compound in the aqueous cannabinoid composition contained in the barrel 102 is higher than the concentration of the at least one vaporizable compound in the dilute vaporizable compound composition and lower than the concentration of the at least one vaporizable compound in the concentrated vaporizable compound composition.

According to some embodiments, upon a sufficient amount of the aqueous cannabinoid and/or nicotine composition being transferred from the barrel 102 to the concentrating module 110, the at least one cannabinoid and/or nicotine undergoes stronger retention to the packing material than the water, thereby a dilute cannabinoid and/or nicotine composition is flowing to the drain chamber 130 through the opening 135 and a concentrated cannabinoid and/or nicotine composition is retained within the concentrating module 110; wherein the concentration of the at least one cannabinoid and/or nicotine in the aqueous cannabinoid composition contained in the barrel is higher than the concentration of the at least one cannabinoid and/or nicotine in the dilute barrel 102 composition and lower than the concentration of the at least one cannabinoid and/or nicotine in the concentrated cannabinoid and/or nicotine composition.

According to some embodiments, the concentrating module 110 has a total volume V_{c}, the packing material has a volume Vₛₚ and the remaining volume, V_{c}-Vₛₚ is free space, wherein upon a volume larger than V_{c}-Vₛₚ of the aqueous composition being transferred from the barrel 102 to the concentrating module 110, the dilute composition is flowing to the drain chamber 130 and the concentrated composition is retained within the concentrating module 110.

According to some embodiments, the aerosol generating device cartridge 100 further comprises a mouthpiece. According to some embodiments, the mouthpiece is located proximally to the outlet 150. According to some embodiments, the aerosol generating device cartridge 100 further comprised a mouthpiece located proximally to the outlet 150.

As detailed below, the concentrating module 110 may include thermally sensitive organic and/or particulate material. Therefore, according to some embodiments, it may be beneficial to incorporate a thermally insulating layer (not shown) between the concentrating module 110 and the evaporative heater 120. According to some embodiments, the aerosol generating device cartridge 100 further comprises a thermally insulating layer located between the concentrating module 110 and the evaporative heater 120. According to some embodiments, the thermally insulating layer is configured to allow passage of fluids therethrough. According to some embodiments, the thermally insulating layer is not including a chromatographic packing material.

Chromatographic Packing Material

According to some embodiments, the concentrating module 110 is designed as a chromatographic separation column. According to some embodiments, the concentrating module 110 is designed as a chromatographic separation column configured to retain the at least one cannabinoid and/or nicotine from the aqueous composition. According to some embodiments, the concentrating module 110 is designed as a chromatographic separation column configured to retain the at least one cannabinoid and/or nicotine from the aqueous composition through interactions between the at least one cannabinoid and/or nicotine and the packing material. According to some embodiments, the concentrating module 110 is designed as a chromatographic separation column, wherein the packing material is a stationary phase chromatographic packing material. The packing material of the present concentrating module 110 is shown in FIGS. 2-8 as stars. Thus, according to some embodiments, there is provided an aerosol generating device cartridge having a proximal end and a distal end, wherein the cartridge comprises: a barrel having a proximal open face and a distal face, wherein the distal face is facing and located proximally to the distal cartridge end, wherein the barrel comprises an aqueous composition comprising at least one vaporizable compound selected from: a cannabinoid, nicotine, or both; a concentrating module having a distal open face and a proximal open face, wherein the concentrating module distal open face is in contact with the barrel, wherein the concentrating module is surrounded by at least one wall and is containing a chromatographic packing material; an outlet at the cartridge proximal end; and at least one evaporative heater configured to generate heat and to evaporate water from a surface thereof, wherein the evaporative heater is located proximally to the concentrating module proximal open face.

As used herein the term “chromatography” refers to a separation technique wherein a mixture comprising an analyte is passed through a stationary phase and separates the analyte from other molecules in the mixture based on differential partitioning between the mobile and stationary phases.

As used herein, the term “HPLC” or “high performance liquid chromatography” refers to liquid chromatography in which the degree of separation is increased by forcing the mobile phase under pressure through a stationary phase, typically a densely packed column.

The term “mobile phase” refers to a solution that is run through a chromatography column. A “mobile phase” can include one or more solvents, such as water or water ethanol mixture. The term “mobile phase” also includes one or more analytes such as cannabinoid(s) and/or nicotine, which are being separated in a column containing the stationary phase.

As used herein, the term “eluent” refers to a mobile phase as it is delivered through a chromatography column.

The term “stationary phase” refers to a phase that comprises particles that comprise an organic or an inorganic material that optionally has an organic moiety bonded to it that renders the surface of the particle useful in certain chromatographic separations. In certain instances, these particles or materials are fixed in a column and do not move. Examples of inorganic materials are, but are not limited to, silica, zirconium and titanium oxides. An example of an organic material is styrene divinyl benzene copolymer. An inorganic material can also include what are known in the art as “hybrid” particles that contain organic and inorganic moieties in the same structure

The term “retention time” is used in the field of chromatography to mean the amount of time required for a component to elute through a column in a chromatographic separation.

The term “retention” and “retain” as used herein refer to their meaning in chromatography (e.g., in gas chromatography, HPLC, etc.). Specifically, these terms are not to be construed as absolute, but relative. In particular, if a mixture of components A and B passes through a column containing a stationary phase, and the progression rate through the column is higher for component B than it is for component A, component A is regarded to be retained by the stationary phase (although component A may finally complete the passage through the column). Therefore, the phrase “chromatographic separation column configured to retain the at least one cannabinoid and/or nicotine from the aqueous composition” means that the chromatographic separation column ‘holds’ the cannabinoid and/or nicotine stronger than it does the aqueous medium. This is generally achieved through interactions between the at least one cannabinoid and/or nicotine and the packing material.

The term “retention factor” is defined in terms of the measured parameters t_{R} and t₀, where t_{R} is the retention time of the measured peak, and t₀ is retention time of the non-retained component. The retention factor (k) can be calculated from the formula k=(t_{R}-t₀)/t₀. Specifically, if upon elution of a compound from a composition (e.g. a solution) through a the specific stationary phase, the compound is strongly retained, the retention factor will be high.

According to some embodiments, the retention factor of the at least one cannabinoid upon elution of the cannabinoid composition through a column of the packing material is at least 2. According to some embodiments, the at least one cannabinoid-water-packing material retention factor is at least 2.5. According to some embodiments, the at least one cannabinoid-water-packing material retention factor is at least 3. According to some embodiments, the at least one cannabinoid-water-packing material retention factor is at least 3.5. According to some embodiments, the at least one cannabinoid-water-packing material retention factor is at least 4. According to some embodiments, the at least one cannabinoid-water-packing material retention factor is at least 4.5. According to some embodiments, the at least one cannabinoid-water-packing material retention factor is at least 5. According to some embodiments, tetrahydrocannabinol-water-packing material retention factor is at least 2. According to some embodiments, tetrahydrocannabinol-water-packing material retention factor is at least 2.5. According to some embodiments, tetrahydrocannabinol-water-packing material retention factor is at least 3. According to some embodiments, tetrahydrocannabinol-water-packing material retention factor is at least 3.5. According to some embodiments, tetrahydrocannabinol-water-packing material retention factor is at least 4. According to some embodiments, tetrahydrocannabinol-water-packing material retention factor is at least 4.5. According to some embodiments, tetrahydrocannabinol-water-packing material retention factor is at least 5. According to some embodiments, tetrahydrocannabinolic acid-water-packing material retention factor is at least 2. According to some embodiments, tetrahydrocannabinolic acid-water-packing material retention factor is at least 2.5. According to some embodiments, tetrahydrocannabinolic acid-water-packing material retention factor is at least 3. According to some embodiments, tetrahydrocannabinolic acid-water-packing material retention factor is at least 3.5. According to some embodiments, tetrahydrocannabinolic acid-water-packing material retention factor is at least 4. According to some embodiments, tetrahydrocannabinolic acid-water-packing material retention factor is at least 4.5. According to some embodiments, tetrahydrocannabinolic acid-water-packing material retention factor is at least 5. According to some embodiments, tetrahydrocannabinolic acid-water-packing material retention factor is at least 2. According to some embodiments, tetrahydrocannabinolic acid basic salt-water-packing material retention factor is at least 2.5. According to some embodiments, tetrahydrocannabinolic acid basic salt-water-packing material retention factor is at least 3. According to some embodiments, tetrahydrocannabinolic acid basic salt-water-packing material retention factor is at least 3.5. According to some embodiments, tetrahydrocannabinolic acid basic salt-water-packing material retention factor is at least 4. According to some embodiments, tetrahydrocannabinolic acid basic salt-water-packing material retention factor is at least 4.5. According to some embodiments, tetrahydrocannabinolic acid basic salt-water-packing material retention factor is at least 5. According to some embodiments, cannabidiol-water-packing material retention factor is at least 2. According to some embodiments, cannabidiol-water-packing material retention factor is at least 2.5. According to some embodiments, cannabidiol-water-packing material retention factor is at least 3. According to some embodiments, cannabidiol-water-packing material retention factor is at least 3.5. According to some embodiments, cannabidiol-water-packing material retention factor is at least 4. According to some embodiments, cannabidiol-water-packing material retention factor is at least 4.5. According to some embodiments, cannabidiol-water-packing material retention factor is at least 5. According to some embodiments, cannabidiolic acid-water-packing material retention factor is at least 2. According to some embodiments, cannabidiolic acid-water-packing material retention factor is at least 2.5. According to some embodiments, cannabidiolic acid-water-packing material retention factor is at least 3. According to some embodiments, cannabidiolic acid-water-packing material retention factor is at least 3.5. According to some embodiments, cannabidiolic acid-water-packing material retention factor is at least 4. According to some embodiments, cannabidiolic acid-water-packing material retention factor is at least 4.5. According to some embodiments, cannabidiolic acid-water-packing material retention factor is at least 5. According to some embodiments, cannabidiolic acid basic salt-water-packing material retention factor is at least 2. According to some embodiments, cannabidiolic acid basic salt-water-packing material retention factor is at least 2.5. According to some embodiments, cannabidiolic acid basic salt-water-packing material retention factor is at least 3. According to some embodiments, cannabidiolic acid basic salt-water-packing material retention factor is at least 3.5. According to some embodiments, cannabidiolic acid basic salt-water-packing material retention factor is at least 4. According to some embodiments, cannabidiolic acid basic salt-water-packing material retention factor is at least 4.5. According to some embodiments, cannabidiolic acid basic salt-water-packing material retention factor is at least 5.

According to some embodiments, the retention factor of nicotine upon elution of the nicotine composition through a column of the packing material is at least 2. According to some embodiments, the nicotine-water-packing material retention factor is at least 2.5. According to some embodiments, nicotine-water-packing material retention factor is at least 3. According to some embodiments, the nicotine-water-packing material retention factor is at least 3.5. According to some embodiments, the nicotine-water-packing material retention factor is at least 4. According to some embodiments, the nicotine-water-packing material retention factor is at least 4.5. According to some embodiments, the nicotine-water-packing material retention factor is at least 5. According to some embodiments, protonated nicotine-water-packing material retention factor is at least 2. According to some embodiments, protonated nicotine-water-packing material retention factor is at least 2.5. According to some embodiments, protonated nicotine-water-packing material retention factor is at least 3. According to some embodiments, protonated nicotine-water-packing material retention factor is at least 3.5. According to some embodiments, protonated nicotine-water-packing material retention factor is at least 4. According to some embodiments, protonated nicotine-water-packing material retention factor is at least 4.5. According to some embodiments, protonated nicotine-water-packing material retention factor is at least 5.

It is to be understood that nicotine may be protonated, based on the pH of the aqueous composition. Thus, “nicotine” according to the present disclosure, relates to basic nicotine or protonated nicotine. It is further to be understood that for cartridge according to the present invention, which contain protonated nicotine, the packing material has higher affinity to protonated nicotine compound than its affinity to water.

According to some embodiments, the packing material is a chromatographic stationary phase packing material. According to some embodiments, the packing material is selected from the group consisting of: activated carbon, an ion exchange chromatography stationary phase packing material, a reversed-phase chromatography stationary phase packing material, a size exclusion chromatography stationary phase packing material and a combination thereof.

According to some embodiments, the chromatographic stationary phase packing material comprises silica, alumina, zirconia, titania, a cross linked polymer, a derivative or combination thereof. According to some embodiments, the chromatographic stationary phase packing material comprises a solid support material selected from silica, alumina, zirconia, titania, a cross linked polymer, derivatives and combination thereof. The term “derivative” as used herein is to be understood from the below description, and include any chemical or physical modification performed on the solid support material. For example, silica C18 is a silica derivative.

As appreciated by the skilled in the art, small, spherical particles of stationary phase packing material are available in a variety of particle diameters (1.5-10 μm) and pore sizes (60-4000 A) of narrow distribution. According to some embodiments, the stationary phase packing material consists of particle diameter in the range of 1.5-10 μm, including each value and/or sub-range within this range. For example, according to some embodiments, the stationary phase packing material consists of particle diameter in the range of 1.5-3 μm, 3-5 μm, 5-7.5 μm, 7.5-10 μm and the like. According to some embodiments, the stationary phase packing material is porous. According to some embodiments, the stationary phase packing material has pore size in the range of 60-4000 Angstrom, including each value and/or sub-range within this range. For example, according to some embodiments, the stationary phase packing material has pore size in the range of 60-100, 100-200, 200-400, 400-600, 600-1000, 1000-2000 or 2000-400 Angstrom.

In addition, the solid support (e.g. silica or alumina) surface can be easily modified through its reactive MOH (i.e. SiOH or AlOH) sites. This is employed mainly, but not limited, in reversed-phase liquid chromatography packing materials. According to some embodiments, the packing material consists of a chemically modified solid support. According to some embodiments, the packing material consists of a solid support chemically bonded to an hydrophobic species. According to some embodiments, the solid support is selected from the group consisting of silica, alumina, zirconia and titania. According to some embodiments, the solid support is silica.

There are three sorts of silica used for the preparation of chromatograhic packings: (i) spherical and irregular particles; (ii) pellicular particles, (iii) multiporous channels. According to some embodiments, the silica is selected from the group consisting of: spherical irregular particles; pellicular particles and multiporous channels. Each possibility represents a separate embodiment. According to some embodiments, the silica is a porous silica. According to some embodiments, the silica is a non-porous silica.

According to some embodiments, the of silica solid support is prepared by a method selected from acid hydrolysis of inorganic silicates; and hydrolysis of alkoxysilans. Each possibility represents a separate embodiment of the invention.

Reversed-Phase Chromatography Packing Material

According to some embodiments, the packing material is a reversed-phase chromatography packing material.

Reversed-phase liquid chromatography (RP-HPLC) is a widely employed technique in the separation and analysis of a great variety of compounds with different chemical functionalities. The most common type of stationary phase for RP-HPLC has a packing material consisting of nonpolar, hydrophobic organic species (e.g., octyl, octadecyl) attached by siloxane bonds to the surface of a silica support.

However, reverse-phase stationary packings according to the present invention are not limited to C₈₋₁₈-silica silica, a great variety of stationary phase packings are commercially available and allow to achieve the desired selectivity

According to some embodiments, the packing material consists of a solid support chemically bonded to an hydrophobic species. According to some embodiments, the solid support is selected from the group consisting of silica, alumina, zirconia and titania. According to some embodiments, the solid support is silica.

According to some embodiments, the packing material is represented by Formula I:

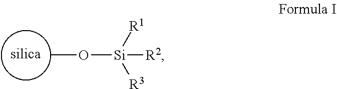

wherein
each one of R¹, R² and R³ is independently selected from the group consisting of a (C₁-C₃₀)-alkyl, a cycloalkyl, an aryl and a heteroaryl, each of which if optionally substituted with at least one substituent selected from the group consisting of halogen, nitro (NO₂), (C₁-C₃₀)-alkyl, C₂-C₃₀ alkenyl, C₂-C₃₀ alkynyl, cycloalkyl, aryl, heterocyclyl, heteroaryl, (C₁-C₃₀)-alkylaryl, (C₁-C₃₀)-alkylcycloalkyl, (C₁-C₃₀)-alkylheterocyclyl, (C₁-C₃₀)-alkylheteroaryl, haloalkyl, hydroxy (OH), (C₁-C₃₀)-alkyloxy, aryloxy, thio (SH), (C₁-C₃₀)-alkylthio, arylthio, —C(O)R, —C(O)OR, —X—(CH₂)ₜ-G, amino (NH₂), —NRR′, —C(O)NRR′, —S(O)OR, —S(O)₂OR, —S(O)NRR′ and —S(O)₂NRR′;
R and R′ are each independently H, (C₁-C₃₀)-alkyl, cycloalkyl or an aryl; and wherein each of said alkyl, cycloalkyl, heterocyclyl, aryl and heteroaryl is optionally substituted;
X is a bond, O, S or NH;
G is an acidic or basic moiety; and
each one of n and t is independently an integer in the range of 0 to 30;
and salts thereof.

According to some embodiments, each one of R¹, R² and R³ is independently selected from the group consisting of a (C₁-C₃₀)-alkyl, a cycloalkyl, an aryl and a heteroaryl. According to some embodiments, each one of R¹, R² and R³ is independently selected from the group consisting of a (C₁-C₃₀)-alkyl, a cycloalkyl, and an aryl. According to some embodiments, each one of R¹, R² and R³ is independently a (C₁-C₃₀)-alkyl. According to some embodiments, each one of R¹, R² and R³ is independently an unsubstituted (C₁-C₃₀)-alkyl. According to some embodiments, each one of R¹, R² and R³ is independently an unsubstituted (C₁-C₂₅)-alkyl. According to some embodiments, each one of R¹, R² and R³ is independently an unsubstituted (C₁-C₂₀)-alkyl.

According to some embodiments, at least one of R¹, R² and R³ is an unsubstituted (C₄-C₃₀)-alkyl. According to some embodiments, at least one of R¹, R² and R³ is an unsubstituted (C₄-C₂₅)-alkyl According to some embodiments, at least one of R¹, R² and R³ is an unsubstituted (C₄-C₂₀)-alkyl. According to some embodiments, at least one of R¹, R² and R³ is an unsubstituted (C₄-C₃₀)-alkyl. According to some embodiments, at least one of R¹, R² and R³ is an unsubstituted (C₈-C₁₈)-alkyl. According to some embodiments, at least one of R¹, R² and R³ is selected from the group consisting of C₈H₁₇ and C₁₈H₃₇. According to some embodiments, at least one of R¹, R² and R³ is C₁₈H₃₇.

According to some embodiments, at least one of R¹, R² and R³ is an unsubstituted (C₁-C₄)-alkyl. According to some embodiments, at least one of R¹, R² and R³ is an unsubstituted (C₁-C₂)-alkyl. According to some embodiments, at least one of R¹, R² and R³ is a methyl.

It is to be understood that when unsubstituted alkyls are referred, either as a short formula, e.g. C₁, C₂, C₄, C₈, C₁₈, C₂₀, C₃₀, etc., or as a complete formula, it is refereed to either a linear alkyl or a non-linear alkyl.

According to some embodiments, each one of R¹ and R² is an unsubstituted (C₁-C₄)-alkyl and R³ is an unsubstituted (C₄-C₃₀)-alkyl. According to some embodiments, each one of R¹ and R² is an unsubstituted (C₁-C₂)-alkyl and R³ is an unsubstituted (C₄-C₂₀)-alkyl. According to some embodiments, each one of R¹ and R² is CH₃ and R³ is an unsubstituted (C₈-C₁₈)-alkyl. According to some embodiments, each one of R¹ and R² is CH₃ and R³ is C₈H₁₇. According to some embodiments, each one of R¹ and R² is CH₃ and R³ is C₁₈H₃₇.

Alkyl chains of different lengths are the groups currently used in liquid chromatography, and may be included in the packing of the present invention, according to some embodiments. Among such alkyl chains, octyl (either n-octyl or ethyl-2-hexyl) and n-octadecyl represent most available HPLC packings. But bonded silicas with other chain lengths are also available, and contemplated as the packing material of the present invention. For example, methyl, ethyl, propyl, butyl, hexyl, dodecyl (Permacoat RP 12 ™, Synergi MAX Rp™), tetradecyl (Chromatem C14™ or Kovasil C14™), dococyl (Chromegabond C22™ or Techsil C22™) and triacontyl (YMC C30™). Reversed phases of shorter chain length, from C1 to C4, are also contemplated.

Groups such as phenyl, phenyl 3-propyl, diphenyl 3 3 propyl, phenyl 6-hexyl (Luna phenylhexyl™), pyrenyl 3-propyl (Cosmosil PYE™), cyclohexa 3-propyl, octancarbamido 3-propyl, octadeca-amidyl 3-propyl (Nucleosil Protect™), octadecyl dimethyl N, N, N, N, 3-propyl (Stability BS-C23™), (stearyl-3-glycidoxy 1)-3-propyl, heptadecafluoroocta 2-ethyl (Fluofix™), and cyano 3-propyl, which are also available for RPLC from different manufacturers, are also to be considered as packing materials for the present invention. Polyfluorinated bonded silicas, polyaromatic hydrocarbon bonded silicas and cyanoalkyl bonded silicas are also to be considered as packing materials for the present invention.

The synthesis of chemically bonded silica supports can be performed in two main ways, as detailed below. similar syntheses may be carried out to prepare reverse phase packing and other packings. Typically, silica chemically bonded to organic chains is used in reverse phase chromatography and can be performed in two ways:

1. Bonding is carried out during the formation of the porous support. Organohalo-or alkoxysilanes are commonly employed as starting materials and are converted by hydrolysis, condensation and polymerisation into rigid, insoluble and porous polyorganosiloxanes or organosilicon xerogels. In the final product, the organofunctional groups are distributed on the surface.

2. Surface modification—a method which involves a reaction between a porous silica and an appropriate reactive organosilane —R¹R²R³SiX, wherein X is a halogen or an oxygen containing leaving group and R¹, R² and R³ are as described above.

According to some embodiments, the packing material is a silica chemically bonded to organic chains, and is prepared by a method selected from bonding out during the formation of the porous support; and surface modification.

Stationary phases other than silica bonded to side chain are also contemplated.

According to some embodiments, the packing material is a polymer based stationary phase. According to some embodiments, the polymer is a copolymer. According to some embodiments, the polymer is polystyrene divinylbenzene.

The most employed polymer as a solid phase packing material for HPLC is polystyrene divinylbenzene (PS-DVB) (PolyRP™, PRP™ and Polysphere™). The chromatographic retention behavior of similar compounds on this support is similar to that of C18 silica-based stationary phases. However, aromatic solutes are more strongly retained due to the π-π interactions between the support and compounds containing an aromatic group. A polyvinyl pyridine-polystyrene polymer has also been reported. It is hypothesized that such π-π interaction may be beneficial to provide strong retention of the aromatic cannabinoids and nicotine, thereby to provide a concentrated aqueous composition of cannabinoids and/or nicotine upon passing of the aqeuous composition through the concentrating module 110, as described herein.

Other polymers can also be used as packing materials according to the present invention. These include a polyvinyl alcohol support bonded with a stearyl chain, which was reported for HPLC (Asahipack ODP-50™).

According to some embodiments, the packing material is a polymer-coated silica-based phase.

A polymer-coated silica-based phase is formed by bonding a polymeric layer on a silica support. This combines the mechanical stability of a silica support with the chemical stability of organic polymer particles. The polymer layer of such packing material may, without limitation, be either be physically adsorbed or chemically bonded. One example of a polymer-coated silica-based phase, which may be used for the present packing material, is polybutadiene (PBD) coated silica support. Another example contemplated herein is polyhydroxyethylaspartamide-silica, which was used as a hydrophilic polymer for eluting polar compounds.

Although silica is the most prevalent support, other metal oxides are also contemplated for use part of the packing material of the present invention.

Known metal oxides used for HPLC include alumina (aluminium oxide), zirconia (zirconium dioxide), thoria (thorium dioxide), magnesia (magnesium oxide) and titania (titanium dioxide).

Mixed oxide gels are also contemplated. Known mixed metal oxides used for HPLC include such as silica-zirconia, silica-alumina, silica-titania and silica-magnesia.

It is to be understood that each of the non-silica supports detailed above may be chemically modified in order to provide the desired separation and stability properties. Such modifications are detailed with respect to silica resins, as these are the most common, however, the modifications may similarly apply to alumina, magnesia, thoria, titania, zirconia and mixed oxides thereof.

According to some embodiments, the packing material is a Porous Graphitic Carbon (PGC).

Porous Graphitic Carbon (PGC) is composed entirely of sheets of hexagonal arranged carbon atoms showing sp² hybridization. PGC has regular, homogeneous, flat surface and random, amorphous surface, which is associated with conventional silica-based stationary phases.

In reverse-phase liquid chromatography, which is now one of the most common forms of liquid chromatography, the stationary phase is less polar than the mobile phase. For example, aqueous compositions of organic compounds, such as the aqueous cannabinoid composition within the aerosol generating device cartridge 100 and aerosol generating device 200 of the present invention, may pass through the concentrating module 110, which is packed with reverse-phase liquid chromatography packing material. Upon such passage, the less polar cannabinoids are substantially retained in the concentrating module 110, whereas the more polar water is passed at a faster rate through the concentrating module 110. Specifically, since both the reverse-phase liquid chromatography packing material and the cannabinoids are relatively non-polar they form hydrophobic interactions, which attract the cannabinoids to the solid phase, while such hydrophobic interactions are not formed between water and the solid phase packing. In contrast, since the reverse-phase liquid chromatography packing material is typically hydrophobic (i.e. water-repelling), water is repelled thereby. This results in increased rate of passage of water and decreased rate of passage of water in the concentrating module 110, and to an increase of the cannabinoid concentration therein, as a result.

Carbon-Based Chromatographic Support Materials

According to some embodiments, the packing material is a carbon-based chromatographic support material. According to some embodiments, the packing material comprises activated carbon. According to some embodiments, the packing material is activated carbon.

Carbon particles, such as those referred to as “activated carbon,” are employed in sorbent applications due to their relatively high specific sorption capacity. This capacity is at least partially due to carbon's low density and the fact that it can be made highly porous.

Carbon has also been employed in chromatographic applications because it offers a hydrophobic and hydrophilic selectivity which is different than that of the silica supports commonly utilized in reversed-phase HPLC. Carbon's selectivity to polar compounds also varies from the selectivities of conventional HPLC packing materials. These differences in selectivity can be advantageously exploited.

A further advantage of carbon-based supports is their pH stability. This stability allows separations to be performed at the optimal pH, and also permits cleaning and sterilizing of the column with, for example, strong base.

Packing materials for high pressure liquid chromatography (HPLC) have also been based on carbon. For example, carbon-based supports useful for HPLC applications have included the following: graphitized carbon black (GCB), pyrocarbon reinforced GCB, and more recently, a porous graphitic carbon (PGC). PGC is prepared by filling the pores of a silica gel with a polymer comprising carbon, thermolyzing the polymer to produce a silica/carbon composite, dissolving out the silica to produce a porous carbon, and subjecting the porous carbon to graphitizing conditions.

According to some embodiments, the packing material is selected from the group consisting of graphitized carbon black (GCB), pyrocarbon reinforced GCB and a porous graphitic carbon (PGC).

For example, U.K. Patent Application No. 2,035,282 discloses a method for producing a porous carbon material suitable for chromatography or use as a catalyst support, which involves depositing carbon in the pores of a porous inorganic template material such as silica gel, porous glass, alumina or other porous refractory oxides having a surface area of at least 1 m2/g, and thereafter removing the template material. O. Chiantore et al., Analytical Chemistry, 60, 638-642 (1988), disclose carbon sorbents which were prepared by pyrolysis of either phenol formaldehyde resin or saccharose on spheroidal silica gels coated with these materials. The pyrolysis is performed at 600° C. for one hour in an inert atmosphere, and the silica is subsequently removed by boiling the material in an excess of a 10% NaOH solution for 30 minutes.

The use of pyrocarbon-reinforced carbon-based supports for HPLC is also known. For example, K. Unger et al. (U.S. Pat. No. 4,225,463) disclose porous carbon support materials based on activated carbons and/or cokes, which may be useful for HPLC. The materials are prepared by treating hard activated carbon or coke particles with solvents, and then heating them at 2400°−3000° C. under an inert gas atmosphere. The resulting support materials are disclosed as having a carbon content of at least 99 percent, a specific surface area of about 1-5 m2 per gram, and a particle size of about 5-50 m.

Ion Exchange Chromatography Packing Material

According to some embodiments, the packing material is an ion exchange chromatography packing material. According to some embodiments, the ion exchange chromatography packing material is selected from the group consisting of an anion exchange chromatography packing material and cation exchange chromatography packing material. According to some embodiments, the ion exchange chromatography packing material is a cation exchange chromatography packing material. According to some embodiments, the ion exchange chromatography packing material is an anion exchange chromatography packing material.

In general, most typical ion-exchange resins are based on crosslinked polystyrene. The actual ion-exchanging sites are introduced after polymerization. Additionally, in the case of polystyrene, crosslinking is introduced by copolymerization of styrene and a few percent of divinylbenzene. Crosslinking decreases ion-exchange capacity of the resin and prolongs the time needed to accomplish the ion-exchange processes but improves the robustness of the resin. Particle size also influences the resin parameters; smaller particles have larger outer surface, but cause larger head loss in the column processes.

Four main types of ion-exchange resins differ in their functional groups:
strongly acidic, typically featuring sulfonic acid groups, e.g. sodium polystyrene sulfonate or polyAMPS,
strongly basic, typically featuring quaternary amino groups, for example, trimethylammonium groups, e.g. polyAPTAC (poly (acrylamido-N-propyltrimethylammonium chloride)) and Colestyramine
weakly acidic, typically featuring carboxylic acid groups,
weakly basic, typically featuring primary, secondary, and/or tertiary amino groups, e.g. polyethylene amine.

A multitude of different mediums are used for the stationary phase. Among the most common immobilized charged groups used are trimethylaminoethyl (TAM), triethylaminoethyl (TEAE), diethyl-2-hydroxypropylaminoethyl (QAE), aminoethyl (AE), diethylaminoethyl (DEAE), sulpho (S), sulphomethyl (SM), sulphopropyl (SP), carboxy (C), and carboxymethyl (CM).

Resins (often termed ‘beads’) of ion exchange columns may include functional groups such as weak/strong acids and weak/strong bases. There are also special columns that have resins with amphoteric functional groups that can exchange both cations and anions. Some examples of functional groups of strong ion exchange resins are quaternary ammonium cation (Q), which is an anion exchanger, and sulfonic acid (S, —SO₂OH), which is a cation exchanger. These types of exchangers can maintain their charge density over a pH range of 0-14. Examples of functional groups of Weak ion exchange resins include diethylaminoethyl (DEAE, —C₂H₄N(CH₂H₅)₂), which is an anion exchanger, and carboxymethyl (CM, —CH₂—COOH).

As known in the art many ion exchange chromatography packing materials are based on a polymeric matrix, which is chemically bonded to an active group, which creates the ionic interaction, which are responsible for the chromatographic separation. According to some embodiments, the ion exchange chromatography packing material comprises a matrix chemically bonded to an active group.

According to some embodiments, the matrix is selected from styrene-divinylbenzene copolymer and an acrylic copolymer.

According to some embodiments, the matrix is selected from styrene-divinylbenzene copolymer gel, macromolecular styrene-divinylbenzene copolymer, macromolecular acrylic copolymer and acrylic gel.

According to some embodiments, the active group is selected from the group consisting of: quaternary ammonium functional group, benzyldimethyl(2-hydroxyethyl)ammonium functional group, dimethylethanolbenzyl ammonium, dimethylethanolamine, trimethylbenzylammonium dimethylethanolamine functional group and trimethylammonium. According to some embodiments, the active group is selected from the group consisting of: tertiary amine, polyamine, N-methylglucamine free base and dimethylamino. According to some embodiments, the active group is selected from the group consisting of: tertiary amine, polyamine, N-methylglucamine free base, dimethylamino, quaternary ammonium functional group, benzyldimethyl(2-hydroxyethyl)ammonium functional group, dimethylethanolbenzyl ammonium, dimethylethanolamine, trimethylbenzylammonium dimethylethanolamine functional group and trimethylammonium.

Size Exclusion Chromatography Packing Material

According to some embodiments, the packing material is a size exclusion chromatography packing material.

Size-exclusion chromatography (SEC), also known as molecular sieve chromatography, is a chromatographic method in which molecules in solution are separated by their size, and in some cases molecular weight. It is usually applied to large molecules or macromolecular complexes such as proteins and industrial polymers. Typically, when an aqueous solution is used to transport the sample through the column, the technique is known as gel-filtration chromatography, versus the name gel permeation chromatography, which is used when an organic solvent is used as a mobile phase. The chromatography column is packed with fine, porous beads which are composed of dextran polymers (Sephadex), agarose (Sepharose), or polyacrylamide (Sephacryl or BioGel P). According to some embodiments, the packing material is selected from the group consisting of dextran polymers, agarose and polyacrylamide.

Aqueous Cannabinoid Composition

According to some embodiments, the aqueous composition is an aqueous cannabinoid composition. According to some embodiments, the aqueous cannabinoid composition is selected from the group consisting of an aqueous cannabinoid solution, an aqueous cannabinoid emulsion and an aqueous cannabinoid suspension. Each possibility represents a separate embodiment of the invention. According to some embodiments, the aqueous cannabinoid composition is an aqueous cannabinoid solution.

According to some embodiments, the aqueous cannabinoid composition has a pH of at least 9.

According to some embodiments, the aqueous cannabinoid composition is consisting of the aqueous solution.

The term “cannabinoid”, as used herein, includes all major and minor cannnabinoids found in natural cannabis and hemp material that can be isolated from a natural source or reproduced by synthetic means. This includes delta-9-Tetrahydrocannabinol (THC), delta-9-Tetrahydrocannabinolic acid (THCA), delta-8-Tetrahydrocannabinol, Cannabidiol (CBD), Cannabidiolic acid (CBDA), Cannabinol (CBN), Cannabinolic acid (CBNA), tetrahydrocannabinovarin (THCV), cannabidivarin (CBDV), cannabigerol (CBG), cannabigerolic acid (CBGA) and cannabichromene (CBC). The term “cannabinoid” also includes basic salts of the acid mentioned above, for example, THCA-sodium salt and THCA-potassium salt.

The term “tetrahydrocannabinolic acid” and “THAC acid” are interchangeable and refer to common derivatives of THC, which are substituted in position 2 of the aromatic ring by a carboxylic acid. THC has two dominant isomers, Δ⁹-THC and Δ⁸-THC. Accordingly, THCA has corresponding Δ⁹ and Δ⁸ isomers. The chemical structures of the parent tetrahydrocannabinols (Δ⁹-THC and Δ⁸-THC) and tetrahydrocannabinolic acids (Δ⁹-THCA and Δ⁸-THCA) are presented below:

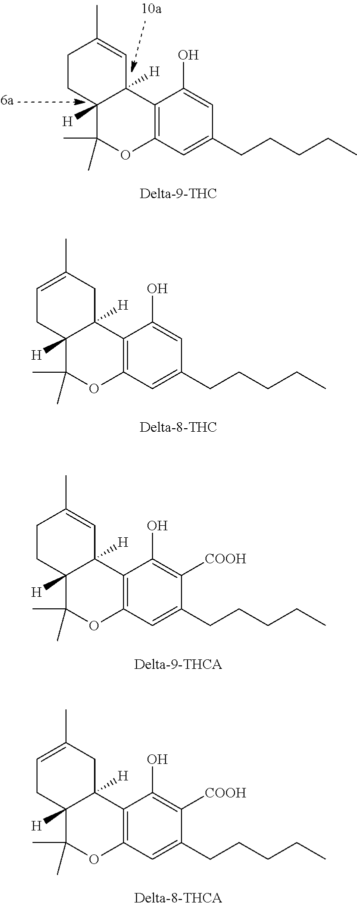

It is to be understood that although the natural THC isomers include an n-C₅H₁₁ chain in position 3, derivatives of THC may include other substituents. Therefore, the term tetrahydrocannabinolic acid includes corresponding structures, in which position 3 is substituted by a group, which is either an n-C₅H₁₁ or a different chemical group.

The term “tetrahydrocannabinolic acid” should be interpreted broadly referring to all possible stereoconfigurations and salts of the relevant formula. Specifically, the natural THC includes two vicinal asymmetric positions, position 6a and position 10a, as shown above. The two vicinal asymmetric positions exist in trans relative configuration, and both are designated R absolute configuration. Thus, the (6aR,10aR) absolute configuration is the preferred configuration for tetrahydrocannabinolic acids of the current invention, however, said tetrahydrocannabinolic acids are not limited to this configuration According to some embodiments, positions 6a and 10a of the THCA acid are in trans relative configuration. According to some embodiments, position 6a has R absolute configuration. According to some embodiments, position 10a has R absolute configuration. According to some embodiments, the THCA acid has the (6aR,10aR) absolute configuration.

As used herein the terms “formulation” and “compositions” generally refer to any mixture, solution, suspension or the like that contains an active ingredient, such as cannabinoid, and, optionally, a carrier. The carrier may be any carrier acceptable for smoking, that is compatible for delivery with the active agent.

According to some embodiments, the administration of the cannabinoid via inhalation comprises generating an inhalable aerosol of the aqueous cannabinoid composition.

According to some embodiments, the administration of the cannabinoid via inhalation comprises generating an inhalable aerosol of the cannabinoid composition upon heating the cannabinoid composition in an aerosol generating device comprising the aerosol generating device cartridge 100 of the present invention.

According to some embodiments, the inhalable aerosol has a pH in the range of 5.5 to 8.5. According to some embodiments, the inhalable aerosol has a pH in the range of 6.0 to 7.5. According to some embodiments, the inhalable aerosol has a pH in the range of 6.5 to 7.5.

It is to be understood to a person skilled in the art that THCA is an organic acid, and thus is better soluble in water, when the pH is elevated. Specifically, at higher (more basic) pH organic acids are present as salts, which are typically more water soluble then their corresponding acids.

According to some embodiments, the aqueous cannabinoid composition has a pH of at least 9.5. According to some embodiments, the aqueous cannabinoid composition has a pH of at least 10. According to some embodiments, the aqueous cannabinoid composition has a pH of at least 10.5. According to some embodiments, aqueous cannabinoid composition has a pH in the range of 9.5 to 11.5. According to some embodiments, aqueous cannabinoid composition has a pH in the range of 9 to 11. According to some embodiments, aqueous cannabinoid composition has a pH in the range of 10 to 11. According to some embodiments, aqueous cannabinoid composition has a pH in the range of 10.5 to 11.5.

According to some embodiments, the concentration of the at least one cannabinoid compound in the aqueous cannabinoid composition is in the range of 2% to 20% w/w. According to some embodiments, the concentration of the at least one cannabinoid compound in the cannabinoid composition is in the range of 5% to 15% w/w. According to some embodiments, the aqueous cannabinoid composition comprises at least one cannabinoid at a concentration of at least 1% w/w. According to some embodiments, the aqueous cannabinoid composition comprises at least one cannabinoid at a concentration of at least 2% w/w. According to some embodiments, the aqueous cannabinoid composition comprises at least one cannabinoid at a concentration of at least 3% w/w. According to some embodiments, the aqueous cannabinoid composition comprises at least one cannabinoid at a concentration of at least 4% w/w. According to some embodiments, the aqueous cannabinoid composition comprises at least one cannabinoid at a concentration of at least 5% w/w. According to some embodiments, the aqueous cannabinoid composition comprises at least one cannabinoid at a concentration of at least 6% w/w. According to some embodiments, the aqueous cannabinoid composition comprises at least one cannabinoid at a concentration of at least 7% w/w. According to some embodiments, the aqueous cannabinoid composition comprises at least one cannabinoid at a concentration of at least 8% w/w. According to some embodiments, the aqueous cannabinoid composition comprises at least one cannabinoid at a concentration of at least 9% w/w. According to some embodiments, the aqueous cannabinoid composition comprises at least one cannabinoid at a concentration of at least 10% w/w.

According to some embodiments, the percentage of the at least one cannabinoid compound in the cannabinoid composition is within the range of 0.1 to 15% w/w. According to some embodiments, the percentage of the at least one cannabinoid compound in the cannabinoid composition is within the range of 0.5 to 12% w/w. According to some embodiments, the percentage of the at least one cannabinoid compound in the cannabinoid composition is within the range of 1 to 10% w/w. According to some embodiments, the percentage of the at least one cannabinoid compound in the cannabinoid composition is within the range of 2 to 8% w/w. According to some embodiments, the percentage of the at least one cannabinoid compound in the cannabinoid composition is within the range of 2.5 to 7.5% w/w. According to some embodiments, the percentage of the at least one cannabinoid compound in the cannabinoid composition is within the range of 3 to 7% w/w. According to some embodiments, the percentage of the at least one cannabinoid compound in the cannabinoid composition is within the range of 3.5 to 6.5% w/w. According to some embodiments, the percentage of the at least one cannabinoid compound in the cannabinoid composition is within the range of 4 to 6% w/w. According to some embodiments, the percentage of the at least one cannabinoid compound in the cannabinoid composition is within the range of 4.5 to 5.5% w/w. According to some embodiments, the percentage of the at least one cannabinoid compound in the cannabinoid composition is about 5% w/w.

As used herein, the term “about” refers to a range of values ±20%, or ±10% of a specified value. For example, the phrase “the percentage is about 5% w/w “includes ±20% of 5, or from 4% to 6%, or from 4.5% to 5.5%.

As used herein, when relating to cannabinoid/nicotine percentages in liquid compositions, unless specified otherwise, the volume ratio, or w/w % is referred. For example, the phrase “the percentage of the at least one cannabinoid is within the range of 4 to 6%” refers to a liquid solution, in which a single weight unit of the solution includes from 0.04 to 0.06 the weight unit of cannabinoid. Specifically, adding 5 gr of THCA to 95 gr of water will result in a 100 gr solution of 5% THCA.

According to some embodiments, the concentration of the at least one cannabinoid in the cannabinoid composition is within the range of 0.5 to 200 mg/ml. According to some embodiments, the concentration of the at least one cannabinoid in the cannabinoid composition is within the range of 1 to 150 mg/ml. According to some embodiments, the concentration of the at least one cannabinoid in the cannabinoid composition is within the range of 2.5 to 100 mg/ml. According to some embodiments, the concentration of the at least one cannabinoid in the cannabinoid composition is within the range of 10 to 100 mg/ml. According to some embodiments, the concentration of the at least one cannabinoid in the cannabinoid composition is within the range of 20 to 90 mg/ml According to some embodiments, the concentration of the at least one cannabinoid in the cannabinoid composition is within the range of 45 to 55 mg/ml.

According to some embodiments, the at least one cannabinoid is the sole active ingredient in the cannabinoid composition. According to some embodiments, the composition comprises cannabinoid(s) as the only active ingredient.

The term “active ingredient” refers to an agent, active ingredient compound or other substance, or compositions and mixture thereof that provide some pharmacological and/or biological, often beneficial, effect.

According to some embodiments, the cannabinoid composition is a pharmaceutical composition.

According to some embodiments, the cannabinoid composition may comprise one or more active agents, other than cannabinoid(s). According to some embodiments, the one or more active agents include one or more pharmaceutically active agents. According to some embodiments, the one or more active agents are suitable or may be adjusted for inhalation. According to some embodiments, the one or more pharmaceutically active agents are directed for treatment of a medical condition through inhalation.

According to some embodiments, the medical condition is amenable to treatment with THC.

According to some embodiments, the cannabinoid composition further comprises at least one carrier acceptable for inhalation. According to some embodiments, the carrier is stable under basic pH conditions. According to some embodiments, the carrier is water soluble under basic pH conditions. According to some embodiments, the pharmaceutical composition further comprises at least one pharmaceutically acceptable carrier, which is acceptable for inhalation. According to some embodiments, the pharmaceutically acceptable carrier is stable under basic pH conditions. According to some embodiments, the pharmaceutically acceptable carrier is water soluble under basic pH conditions.

According to some embodiments, the cannabinoid composition further comprises at least one stabilizer. According to some embodiments, the stabilizer is stable under basic pH conditions. According to some embodiments, the stabilizer is water soluble under basic pH conditions.

According to some embodiments, the cannabinoid composition further comprises at least one additive selected from the group consisting of a propellant, an anti-coughing agent and a flavorant. According to some embodiments, the cannabinoid composition further comprises at least one additive selected from the group consisting of, an anti-coughing agent and a flavorant. According to some embodiments, the cannabinoid composition further comprises at least one anti-coughing agent. According to some embodiments, the cannabinoid composition further comprises at least one flavorant.

According to some embodiments, the cannabinoid composition further comprises at least one additive at a concentration of 0.1-1% w/w. According to some embodiments, the cannabinoid composition further comprises at least one additive at a concentration of 0.1-0.5% w/w. According to some embodiments, the cannabinoid composition further comprises at least one additive at a concentration of 0.1-0.3% w/w.

According to some embodiments, the additive is approved for use in inhaling solutions. According to some embodiments, the additive is stable at basic aqueous conditions. According to some embodiments, the additive is soluble at basic aqueous conditions.

According to some embodiments, the flavorant is a sweetener. According to some embodiments, the sweetener is selected from the group of artificial sweeteners including saccharine, aspartame, dextrose and fructose.

According to some embodiments, the additive is selected from menthol, eucalyptol, tyloxapol and a combination thereof. According to some embodiments, the additive is selected from menthol, eucalyptol, tyloxapol and a combination thereof, and is present at a concentration of 0.1-0.5% w/w based on the total weight of the cannabinoid composition.

According to some embodiments, the cannabinoid composition further comprises at least one preservative. According to some embodiments, the preservative is selected from the group consisting of benzyl alcohol, propylparaben, methylparaben, benzalkonium chloride, phenylethyl alcohol, chlorobutanol, potassium sorbate, phenol, m-cresol, o-cresol, p-cresol, chlorocresol and combinations thereof.

The term “anti-coughing agent” as used herein refers to an active agent used for the suppression, alleviation or prevention of coughing and irritations and other inconveniencies in the large breathing passages that can, or may, generate coughing. Anti-coughing agent include, but are not limited to antitussives, which are used for which suppress coughing, and expectorants, which alleviate coughing, while enhancing the production of mucus and phlegm. Anti-coughing agents may ease the administration of inhaled aerosols.

According to some embodiments, the at least one anti-coughing agent is selected from expectorants, antitussives or both. According to some embodiments, the at least one anti-coughing agent is selected from the group consisting of menthol, dextromethorphan, dextromethorphan hydrobromide, hydrocodone, caramiphen dextrorphan, 3-methoxymorphinan or morphinan-3-ol, carbetapentane, codeine, acetylcysteine and combinations thereof.

According to some embodiments, the at least one cannabinoid compound comprises tetrahydrocannabinol (THC), tetrahydrocannabinolic acid (THCA), cannabidiol (CBD), cannabidiolic acid (CBDA), salts thereof or a combination thereof. According to some embodiments, the at least one cannabinoid compound comprises THCA or a salt thereof.

According to some embodiments, the at least one cannabinoid compound comprises THCA-salt. According to some embodiments, the at least one cannabinoid compound comprises THCA-sodium salt.

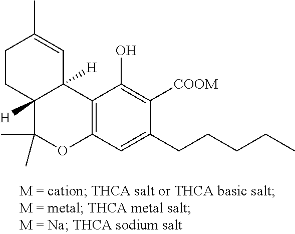

According to some embodiments, the aqueous cannabinoid composition further comprises ethanol. According to some embodiments, the concentration of ethanol is in the range of 0.5-20% v/v.

According to some embodiments, the cannabinoid composition is substantially devoid of organic solvents.

As used herein, “substantially devoid” means that a preparation or composition according to the invention that generally contains less than 3% of the stated substance, such as less than 1% or less than 0.5%.

According to some embodiments, the cannabinoid composition comprises less than 10% w/w organic solvents. According to some embodiments, the cannabinoid composition comprises less than 8% w/w organic solvents. According to some embodiments, the cannabinoid composition comprises less than 6% w/w organic solvents. According to some embodiments, the cannabinoid composition less than 5% w/w organic solvents. According to some embodiments, the cannabinoid composition comprises less than 4% w/w organic solvents. According to some embodiments, the cannabinoid composition comprises less than 3% w/w organic solvents. According to some embodiments, the cannabinoid composition comprises less than 2% w/w organic solvents. According to some embodiments, the cannabinoid composition comprises less than 1% w/w organic solvents. According to some embodiments, the cannabinoid composition comprises less than 0.5% w/w organic solvents.

The heated liquid in conventional electronic cigarettes usually includes a composition or mixture of cannabinoids with humectants, having relatively low latent heat of vaporization, such as propylene glycol (PG) or vegetable glycerin (VG). Said composition is typically referred to as “e-juice”. The liquid mixture is typically drawn into a wicking material that is in contact with a heating element, which may consist a coil of a conducting material to be heated when electric current is driven there through. When not contacted with a liquid, or after the liquid is substantially evaporated the temperature of the coil can reach in some instances a temperature of over 800 degrees Celsius. It is to be understood that in contrast with known e-cigarette compositions, which employ THC formulations comprising PG and/or VG, which are hazardous upon heating and decomposition, the present invention provides aqueous cannabinoid formulations and do not require hazardous organic solvents.

According to some embodiments, the cannabinoid composition is in liquid form. According to some embodiments, the cannabinoid composition comprises at least 40% w/w water. According to some embodiments, the cannabinoid composition comprises at least 50% w/w water. According to some embodiments, the cannabinoid composition comprises at least 60% w/w water. According to some embodiments, the cannabinoid composition comprises at least 70% w/w water. According to some embodiments, the cannabinoid composition comprises at least 75% w/w water. According to some embodiments, the cannabinoid composition comprises at least 80% w/w water. According to some embodiments, the cannabinoid composition comprises at least 85% w/w water. According to some embodiments, the cannabinoid composition comprises at least 90% w/w water. It is to be understood that the phrase “cannabinoid composition comprises at least 90% w/w water” means that each gram of the total composition includes at least 900 milligrams of water and at most 100 milligrams of materials other than water. According to some embodiments, the cannabinoid composition comprises more than 90% w/w water.

Aqueous Nicotine Composition

According to some embodiments, the aqueous composition is an aqueous nicotine composition. According to some embodiments, the aqueous nicotine composition is selected from the group consisting of an aqueous nicotine solution, an aqueous nicotine emulsion and an aqueous nicotine suspension. Each possibility represents a separate embodiment of the invention. According to some embodiments, the aqueous nicotine composition is an aqueous nicotine solution.

According to some embodiments, the aqueous cannabinoid composition is consisting of the aqueous solution.

It is to be understood that nicotine may be provided as the basic nicotine compound or as a protonated nicotine salt. As appreciated by the skilled in the art, nicotine is a weak base with a pKa of about 8, meaning that in neutral aqueous compositions a substantial portion exists in a protonated state and at acidic pH, essentially all the nicotine molecules are protonated and exist in their salt state.

According to some embodiments, the concentration of the nicotine in the aqueous nicotine composition is in the range of 0.5% to 20% w/w. According to some embodiments, the concentration of the nicotine in the nicotine composition is in the range of 0.5% to 15% w/w. According to some embodiments, the aqueous nicotine composition comprises nicotine at a concentration of at least 0.25% w/w, at least 0.5% w/w, at least 0.75% w/w, at least 1% w/w, at least 1.25% w/w, at least 1.5% w/w, at least 1.75% w/w, or at least 1.9% w/w. Each possibility represents a separate embodiment of the invention. According to some embodiments, the aqueous nicotine composition comprises nicotine at a concentration of no more than 20% w/w, no more than 15% w/w, no more than 10% w/w, no more than 7.5% w/w, no more than 5% w/w, no more than 4% w/w, no more than 3.5% w/w, no more than 3% w/w, no more than 2.75% w/w, no more than 2.5% w/w, no more than 2.25% w/w or no more than 2.1% w/w, w/w. Each possibility represents a separate embodiment of the invention.

According to some embodiments, the percentage of the nicotine in the nicotine composition is within the range of 0.1 to 15% w/w. According to some embodiments, the percentage of the nicotine in the nicotine composition is within the range of 0.25 to 10% w/w. According to some embodiments, the percentage of the nicotine in the nicotine composition is within the range of 0.5 to 7.5% w/w. According to some embodiments, the percentage of the nicotine in the nicotine composition is within the range of 1 to 5% w/w. According to some embodiments, the percentage of the nicotine in the nicotine composition is within the range of 1 to 4% w/w. According to some embodiments, the percentage of the nicotine in the nicotine composition is within the range of 1.25 to 3% w/w. According to some embodiments, the percentage of the nicotine in the nicotine composition is within the range of 1.5 to 2.5% w/w. According to some embodiments, the percentage of the nicotine in the nicotine composition is within the range of 1.75 to 2.25% w/w. According to some embodiments, the percentage of the nicotine in the nicotine composition is about 2% w/w.

According to some embodiments, the nicotine is the sole active ingredient in the nicotine composition. According to some embodiments, the composition comprises nicotine as the only active ingredient.

According to some embodiments, the nicotine composition further comprises at least one additive selected from the group consisting of a propellant, an anti-coughing agent and a flavorant. According to some embodiments, the nicotine composition further comprises at least one additive selected from the group consisting of, an anti-coughing agent and a flavorant. According to some embodiments, the nicotine composition further comprises at least one anti-coughing agent. According to some embodiments, the nicotine composition further comprises at least one flavorant.

According to some embodiments, the nicotine composition further comprises at least one additive at a concentration of 0.1-1% w/w. According to some embodiments, the nicotine composition further comprises at least one additive at a concentration of 0.1-0.5% w/w. According to some embodiments, the nicotine composition further comprises at least one additive at a concentration of 0.1-0.3% w/w.

According to some embodiments, the additive is approved for use in inhaling solutions. According to some embodiments, the additive is stable at basic aqueous conditions. According to some embodiments, the additive is soluble at basic aqueous conditions.

Optional flavorants, sweeteners, additives (e.g., menthol, eucalyptol, tyloxapol etc.), preservatives, anti-coughing agents and their amounts in the present nicotine composition are similar to the ones described above in relation to the cannabinoid composition.

According to some embodiments, the nicotine composition is substantially devoid of organic solvents.

According to some embodiments, the nicotine composition comprises less than 10% w/w organic solvents. According to some embodiments, the nicotine composition comprises less than 8% w/w organic solvents. According to some embodiments, the nicotine composition comprises less than 6% w/w organic solvents. According to some embodiments, the nicotine composition less than 5% w/w organic solvents. According to some embodiments, the nicotine composition comprises less than 4% w/w organic solvents. According to some embodiments, the nicotine composition comprises less than 3% w/w organic solvents. According to some embodiments, the nicotine composition comprises less than 2% w/w organic solvents. According to some embodiments, the nicotine composition comprises less than 1% w/w organic solvents. According to some embodiments, the nicotine composition comprises less than 0.5% w/w organic solvents.

According to some embodiments, the nicotine composition is in liquid form.

According to some embodiments, the nicotine composition comprises at least 40% w/w water.

According to some embodiments, the nicotine composition comprises at least 50% w/w water.

According to some embodiments, the nicotine composition comprises at least 60% w/w water.

According to some embodiments, the nicotine composition comprises at least 70% w/w water.

According to some embodiments, the nicotine composition comprises at least 75% w/w water.

According to some embodiments, the nicotine composition comprises at least 80% w/w water.

According to some embodiments, the nicotine composition comprises at least 85% w/w water.

According to some embodiments, the nicotine composition comprises at least 90% w/w water.

According to some embodiments, the nicotine composition comprises more than 90% w/w water.

According to some embodiments, the aqueous nicotine composition comprises aqueous nicotine and a buffer. According to some embodiments, the buffer is an aqueous buffer.

The term “buffer” is well known as a general description of a solution containing either a weak acid and its salt or a weak base and its salt, which is resistant to changes in pH. For example, the term “citrate buffer” is intended to describe a solution comprising citric acid and deprotonated citrate anion, such as, but not limited to, sodium citrate.

According to some embodiments, the buffer is useful within a pH range of about pH 3 to about pH 5. According to some embodiments, the buffer maintains the pH of a solution in range of about pH 3 to about pH 5. According to some embodiments, the buffer is approved for use in inhaling solutions.

According to some embodiments, the buffer is selected from citrate buffers, phosphate buffers and a combination thereof. According to some embodiments, the buffer comprises a citrate buffer. According to some embodiments, the buffer is a citrate buffer. According to some embodiments, the buffer is a phosphate buffer. According to some embodiments, the buffer comprises a phosphate buffer.

According to some embodiments, the aqueous nicotine composition has pH below 6. According to some embodiments, the aqueous nicotine composition is having a pH within the range of 3.0 to 5.0. According to some embodiments, the aqueous nicotine composition is having a pH within the range of 3.5 to 4.5. According to some embodiments, the aqueous nicotine composition is having a pH of about 4.

According to some embodiments, the nicotine in the nicotine composition has a purity of at least 95%.

As used herein the term “purity” and “pure” relate to the chemical purity of a compound which may contain other chemical compounds as impurities wherein the particular compound is present in an amount of at least about 90%, preferably at least about 95%, more preferably at least about 99%, most preferably at least about 99.5% by weight. Typically, the purity can be measured by HPLC. Specifically, impurities commonly present in compositions of nicotine include its related oxidation side products, such as the dehydrogenated myosmine or the oxygenated cotinine and nicotine-N-oxide. Therefore, the term “nicotine having a purity of at least 95%” is meant to describe a composition in which the nicotine related impurities are present in a weight amount, which constitutes not more than 5% relative to the weight of the nicotine in the composition.

According to some embodiments, the nicotine composition may be included within a pharmaceutical composition. According to some embodiments, the pharmaceutical composition may comprise one or more pharmaceutically active agents, other than nicotine. According to some embodiments, the one or more pharmaceutically active agents are suitable or may be adjusted for inhalation. According to some embodiments, the one or more pharmaceutically active agents are directed for treatment of a medical condition through inhalation.

According to some embodiments, the pharmaceutical composition further comprises at least one pharmaceutical acceptable carrier. In other embodiments, the pharmaceutical composition may further comprise one or more stabilizers.

According to some embodiments, the nicotine aqueous pharmaceutical composition is for treating a disease or disorder in a subject in need thereof.

According to some embodiments, the disorder is nicotine withdrawal syndrome.

According to some embodiments, the subject is having a respiratory disease or disorder. According to some embodiments, the respiratory disease or disorder is a pulmonary disease.

According to some embodiments, the disease is selected from the group consisting of asthma, bronchitis, emphysema, lung infection, cystic fibrosis, AAT deficiency, COPD, ARDS, IRDS, BPD, and MAS. Each possibility is a separate embodiment of the invention.

According to some embodiments, the subject is having a respiratory disease affecting the air ways, the alveoli or the interstitium, such as, asthma, chronic obstructive pulmonary disease, chronic bronchitis, emphysema, acute bronchitis, cystic fibrosis, pneumonia, tuberculosis, fragile connections between alveoli, pulmonary edema, lung cancer in its many forms, acute respiratory distress syndrome, pneumoconiosis, mouth and pharynx cancer, tracheal tumors and interstitial lung disease among others.

Evaporative Heater

According to some embodiments, the evaporative heater 120 is at least partially permeable to the aqueous formulation or wherein the aerosol generating device cartridge 100 comprises a plurality of evaporative heaters 120, wherein upon application of pressure of a fluid against the heater 120 or the heaters, the fluid can pass therethrough.

Without wishing to be bound by any theory or mechanism of action, upon heating of the evaporation heater 120 the aqueous composition is at least partially vaporized into vapor. Subsequently, the vapor in condensed into aerosol, which may be inhaled by a user in need thereof, such as a vaporizer user. As detailed herein, water has high latent heat which may require a strong heater(s), according to some embodiments.

The terms “evaporation heater” and evaporative heater” as use herein are interchangeable and refer to an element, which is configured to generate heat and to evaporate a liquid from a surface thereof. The liquid of the present invention is understood to be the aqueous composition (i.e., the aqueous nicotine and/or cannabinoid(s) composition).

According to some embodiments, the evaporative heater 120 is rigid. According to some embodiments, the evaporative heater 120 is made of metal. According to some embodiments, the evaporative heater 120 has two flat sides, which remain flat when liquid is pressed there through. According to some embodiments, the evaporative heater 120 has a top flat surface and a bottom flat surface, which do not deform when liquid is pressed there through or pressed against at least one of the top surface or the bottom surface.

According to some embodiments, the evaporative heater 120 is configured to provide 3-7 W, 4⁻⁶ W, 4.5-5.9 W, 4.8-5.6 W, 5.0-5.4 W or 5.1-5.3 W per every μl of liquid deposited thereon.

According to some embodiments, the evaporative heater 120 is configured to provide about 5.2 W per every μl of liquid deposited thereon.

According to some embodiments the evaporative heater 120 has heat capacity of no more than 1000 Jkg⁻¹K⁻¹. According to some embodiments, the evaporative heater 120 has heat capacity of no more than 900 Jkg⁻¹K⁻¹. According to some embodiments, the evaporative heater 120 has heat capacity of no more than 800 Jkg⁻¹K⁻¹. According to some embodiments, the evaporative heater 120 has heat capacity of no more than 700 Jkg⁻¹K⁻¹. According to some embodiments, the evaporative heater 120 has heat capacity of no more than 600 Jkg⁻¹K⁻¹.

According to some embodiments, the evaporative heater 120 has a specific heat capacity in the range of 100 to 900 Jkg⁻¹K⁻¹. According to some embodiments, the evaporative heater 120 has a specific heat capacity in the range of 200 to 800 Jkg⁻¹K⁻¹. According to some embodiments, the evaporative heater 120 has a specific heat capacity in the range of 300 to 750 Jkg⁻¹K⁻¹. According to some embodiments, the evaporative heater 120 has a specific heat capacity in the range of 400 to 700 Jkg⁻¹K⁻¹. According to some embodiments, the evaporative heater 120 has a specific heat capacity in the range of 450 to 650 Jkg⁻¹K⁻¹. According to some embodiments, the evaporative heater 120 has a specific heat capacity in the range of 500 to 600 Jkg⁻¹K⁻¹. According to some embodiments, the evaporative heater 120 has a specific heat capacity in the range of 470 to 570 Jkg⁻¹K⁻¹. According to some embodiments, the evaporative heater 120 has a specific heat capacity in the range of 485 to 555 Jkg⁻¹K⁻¹. According to some embodiments, the evaporative heater 120 has a specific heat capacity in the range of 500 to 540 Jkg⁻¹K⁻¹.

According to some embodiments, the evaporative heater 120 has surface heat flux in the range of 170 WCm⁻² to 290 WCm⁻². According to some embodiments, the evaporative heater 120 has surface heat flux in the range of 200 Wcm⁻² to 260 WCm⁻². According to some embodiments, the evaporative heater 120 has surface heat flux in the range of 210 Wcm⁻² to 250 WCm⁻². According to some embodiments, the evaporative heater 120 has surface heat flux in the range of 220 WCm⁻² to 240 WCm⁻². According to some embodiments, the evaporative heater 120 has surface heat flux of about 228 Wcm⁻².

According to some embodiments, the evaporative heater 120 is configured to provide an energy output of at least 35 Joules within half a second. According to some embodiments, the evaporative heater 120 is configured to provide an energy output of at least 40 Joules within half a second. According to some embodiments, the evaporative heater 120 is configured to provide an energy output of at least 45 Joules within half a second. According to some embodiments, the evaporative heater 120 is configured to provide an energy output of at least 50 Joules within half a second. According to some embodiments, the evaporative heater 120 is configured to provide an energy output of at least 51 Joules within half a second.

According to some embodiments, the evaporative heater 120 has a total resistance in the range of 0.10Ω to 0.60Ω. According to some embodiments, the evaporative heater 120 has a total resistance in the range of 0.13Ω to 0.55Ω. According to some embodiments, the evaporative heater 120 has a total resistance in the range of 0.15Ω to 0.5Ω. According to some embodiments, the evaporative heater 120 has a total resistance in the range of 0.15Ω to 0.45Ω. According to some embodiments, the evaporative heater 120 has a total resistance in the range of 0.2Ω to 0.4Ω.

According to some embodiments, the evaporative heater 120 is configured to provide an energy output of at least 30 Watts. According to some embodiments, the evaporative heater 120 is configured to provide an energy output of at least 40 Watts. According to some embodiments, the evaporative heater 120 is configured to provide an energy output of at least 50 Watts. According to some embodiments, the evaporative heater 120 is configured to provide an energy output of at least 60 Watts. According to some embodiments, the evaporative heater 120 is configured to provide an energy output of at least 70 Watts. According to some embodiments, the evaporative heater 120 is configured to provide an energy output of at least 80 Watts. According to some embodiments, the evaporative heater 120 is configured to provide an energy output of at least 90 Watts. According to some embodiments, the evaporative heater 120 is configured to provide an energy output of at least 100 Watts. According to some embodiments, the evaporative heater 120 is configured to provide an energy output of at least 102 Watts.

According to some embodiments, the evaporative heater 120 is configured to drive current in the range of 10A and 40A. According to some embodiments, the evaporative heater 120 is configured to drive current in the range of 15A and 35A. According to some embodiments, the evaporative heater 120 is configured to drive current in the range of 20A and 30A. According to some embodiments, the evaporative heater 120 is configured to drive current in the range of 25A and 30A. According to some embodiments, the evaporative heater 120 is configured to drive current of about 28A.

According to some embodiments, the evaporative heater 120 is disposable. According to some embodiments, the evaporative heater 120 is in the form of a rod, a capsule or a flat disc.

According to some embodiments, the evaporative heater 120 comprises a thermally-conductive material, such as metal.

According to some embodiments, the evaporative heater 120 has thermal mass of not more than 0.3 J/C. According to some embodiments, the evaporative heater 120 has thermal mass of not more than 0.2 J/C. According to some embodiments, the evaporative heater 120 has thermal mass of not more than 0.1 J/C. According to some embodiments, the evaporative heater 120 has thermal mass of less than 0.1 J/C. According to some embodiments, the evaporative heater 120 has thermal mass of less than 0.08 J/C. According to some embodiments, the evaporative heater 120 has thermal mass of less than 0.06 J/C. According to some embodiments, the evaporative heater 120 has thermal mass of less than 0.04 J/C. According to some embodiments, the evaporative heater 120 has thermal mass of less than 0.3 J/C. According to some embodiments, the evaporative heater 120 has thermal mass of less than 0.2 J/C.

According to some embodiments, the evaporative heater 120 has thermal mass in the range of 0.001 J/C to 0.3 J/C. According to some embodiments, the evaporative heater 120 has thermal mass in the range of 0.004 J/C to 0.25 J/C. According to some embodiments, the evaporative heater 120 has thermal mass in the range of 0.006 J/C to 0.2 J/C. According to some embodiments, the evaporative heater 120 has thermal mass in the range of 0.01 J/C to 0.015 J/C.

According to some embodiments, the evaporative heater 120 is made of a uniform material. According to some embodiments, the evaporative heater 120 is made of metal. According to some embodiments, the evaporative heater 120 comprises a metal and/or a metal alloy. According to some embodiments, the evaporative heater 120 comprises a metal alloy. According to some embodiments, the evaporative heater 120 comprises at least one metal selected from iron, nickel, titanium, chromium, aluminum, molybdenum and manganese. According to some embodiments, the alloy comprises at least one metal selected from iron, nickel, titanium, chromium, aluminum, molybdenum, silver, palladium and manganese. Each possibility represents a separate embodiment. According to some embodiments, the evaporative heater 120 comprises a metal having electrical resistivity in the range of 0.3·10⁻⁶ to 3·10·⁻⁶Ω·m at room temperature. According to some embodiments, the evaporative heater 120 comprises a metal having electrical resistivity in the range of 0.4·10⁻⁶ to 2.5·10⁻⁶Ω·m at room temperature. According to some embodiments, the evaporative heater 120 comprises a metal having electrical resistivity in the range of 0.5·10⁻⁶ to 2·10⁻⁶Ω·m at room temperature. According to some embodiments, the evaporative heater 120 comprises a metal having electrical resistivity in the range of 0.6·10⁻⁶ to 1.5·10⁻⁶Ω·m at room temperature. According to some embodiments, the evaporative heater 120 comprises an alloy having electrical resistivity in the range of 0.3·10⁻⁶ to 3·10⁻⁶Ω·m at room temperature. According to some embodiments, the evaporative heater 120 comprises an alloy having electrical resistivity in the range of 0.4·10⁻⁶ to 2.5·10⁻⁶Ω·m at room temperature. According to some embodiments, the evaporative heater 120 comprises an alloy having electrical resistivity in the range of 0.5·10⁻⁶ to 2·10⁻⁶⁻Ω·m at room temperature. According to some embodiments, the evaporative heater 120 comprises an alloy having electrical resistivity in the range of 0.6·10⁻⁶ to 1.5·10⁻⁶Ω·m at room temperature. According to some embodiments, the alloy is selected from Kanthal, Nichrome and stainless steel. According to some embodiments, the alloy is Nichrome. According to some embodiments, the alloy is stainless steel. According to some embodiments, the alloy is 316 L stainless steel.

According to some embodiments, the evaporative heater 120 is configured to generate heat rapidly, such that its temperature elevate rapidly.

According to some embodiments, the evaporative heater 120 is configured to generate heat, to reach a temperature in the range between 50 and 600 degrees Celsius. According to some embodiments, the temperature is at least 95° C., at least 96° C., at least 97° C., at least 98° C., at least 98.5° C., at least 99° C., at least 99.5° C., or at least 100° C. According to some embodiments, the temperature is not more than 600° C., not more than 550° C., not more than 500° C., not more than 450° C., not more than 400° C., not more than 350° C. or not more than 300° C.

According to some embodiments, the evaporative heater 120 comprises a resistive heater. According to some embodiments, the evaporative heater 120 comprises a radio-frequency heater. According to some embodiments, the evaporative heater 120 comprises an induction-coil heater.

Without wishing to be bound by any theory or mechanism of action, the evaporative heater 120 is required to include a relatively strong heater. Specifically, the aerosol generating device cartridge 100 of the present invention is designed to evaporate aqueous compositions, according to some embodiments. Thus, use of water, however, may pose several obstacles. Importantly, water has a high latent heat value, meaning that substantial energy has to be invested in order to evaporate water. Thus, according to some embodiments, the evaporative heater 120 is a strong heater configured to generate enough heat to vaporize an aqueous solution. According to some embodiments, the evaporative heater 120 is configured to generate at least 30 W power. According to some embodiments, the evaporative heater 120 is configured to generate at least 32 W power. According to some embodiments, the evaporative heater 120 is configured to generate at least 34 W power. According to some embodiments, the evaporative heater 120 is configured to generate at least 36 W power. According to some embodiments, the evaporative heater 120 is configured to generate at least 8 W power. According to some embodiments, the evaporative heater 120 is configured to generate at least 40 W power.

An additional obstacle encountered when dealing with evaporation aqueous composition, is the slow evaporation thereof, which stems from the high specific heat capacity of water as well as from the high latent heat of water. Both these high values entail investment of a substantial amount of energy, which in turn, is slower than when using organic formulations (i.e. PG or VG). Thus, together with high electrical power, an additional requirement from the evaporative heater 120 is directed to its low thermal mass. According to some embodiments, the evaporative heater 120 has thermal mass of less than 0.05 J/C.

According to some embodiments, the evaporative heater 120 is configured to generate heat and to evaporate the aqueous composition from the surface thereof to form an aqueous aerosol comprising the at least one vaporizable compound.

According to some embodiments, the evaporative heater 120 is configured to generate heat and to evaporate the aqueous composition from the surface thereof to form an aqueous aerosol comprising the at least one vaporizable compound at a rate of at least 0.25 mg cannabinoids and/or nicotine per second.

Aerosol Generating Device

According to some embodiments, there is provided an aerosol generating device 200 comprising the aerosol generating device cartridge 100 disclosed herein and an actuator 250. According to some embodiments, the actuator 250 is reversibly connectable to the aerosol generating device cartridge 100 at the aerosol generating device cartridge distal end 100d. According to some embodiments, the aerosol generating device 200 further comprises means of transferring at least a portion of the aqueous composition from the barrel 102 to the concentrating module 110.

According to some embodiments, there is provided an aerosol generating device 200 comprising the aerosol generating device cartridge 100 of disclosed herein and an actuator 250, wherein the actuator 250 is reversibly connectable to the aerosol generating device cartridge 100 at the aerosol generating device cartridge distal end 100d, wherein the aerosol generating device 200 further comprises means of transferring at least a portion of the aqueous composition from the barrel 102 to the concentrating module 110.

According to some embodiments, the means of transferring at least a portion of the aqueous composition from the barrel 102 to the concentrating module 110 is selected from a liquid pump and a plunger assembly 210.

According to some embodiments, the means of transferring at least a portion of the aqueous composition from the barrel 102 to the concentrating module 110 comprises a plunger assembly 210.

According to some embodiments, the plunger assembly 210 comprises a plunger actuator 212, a rod 214 and a plunger head 216. According to some embodiments, the rod 214 has a rod proximal end 214p and a rod distal end 214d. According to some embodiments, the rod distal end 214d is connected to the plunger actuator 212. According to some embodiments, the plunger actuator 212 is a solenoid actuator. According to some embodiments, the rod proximal end 214p is connected to the plunger head 216. According to some embodiments, the plunger actuator 212 is configured to dislocate the plunger head 216 in the proximal direction from a first position to a second position. According to some embodiments, the plunger actuator 212 is configured to dislocate the plunger head 216 in the proximal direction from a first position to a second position thereby to force at least a portion of the aqueous composition from the barrel 102 to the concentrating module 110.

According to some embodiments, the plunger assembly 210 comprises a plunger actuator 212, a rod 214 and a plunger head 216, wherein the rod 214 has a rod proximal end 214p and a rod distal end 214d, wherein the rod distal end 214d is connected to the solenoid actuator, and the rod proximal end 214p is connected to the plunger head 216, wherein the plunger actuator 212 is configured to dislocate the plunger head 216 in the proximal direction from a first position to a second position, thereby to force at least a portion of the aqueous composition from the barrel 102 to the concentrating module 110.

According to some embodiments, the solenoid actuator is configured to receive electric current and to generate axial movement of the solenoid plunger head 216 upon receiving the electric current, wherein the axial movement is along an elongation of the aerosol generating device cartridge 100.

According to some embodiments, upon dislocating to the second position, the plunger head 216 is at least partially within the barrel 102. According to some embodiments, upon dislocating to the second position, the plunger head 216 is at least partially dislocated into the barrel 102. According to some embodiments, when the plunger head 216 is in the first position, it is not within the barrel 102.

According to some embodiments, the plunger actuator 212, the rod 214 and the plunger head 216 are disposed within the actuator 250. According to some embodiments, upon connecting the actuator 250 and the aerosol generating device cartridge 100 and upon dislocating the plunger head 216 to said second position, it is within the barrel 102.

According to some embodiments, the plunger actuator 212, the rod 214 and the plunger head 216 are disposed within the actuator 250, wherein upon connecting the actuator 250 and the aerosol generating device cartridge 100 and upon dislocating the plunger head 216 to said second position, it is within the barrel 102.

According to some embodiments, the plunger actuator 212 is disposed within the actuator 250 and the rod 214 and the plunger head 216 are disposed within the aerosol generating device cartridge 100.

According to some embodiments, the aerosol generating device 200 further comprises a plunger seal 104 sealingly movable through at least an interior portion of the barrel 102. According to some embodiments, the portion comprises at the barrel distal open face 102d, wherein the plunger seal 104 is in contact with the aqueous composition. According to some embodiments, the portion comprises at the barrel distal open face 102d, wherein the plunger seal 104 is in contact with the aqueous composition thereby sealing and preventing leakage thereof through the barrel distal open face 102d.

According to some embodiments, the aerosol generating device 200 further comprises a plunger seal 104 sealingly movable through at least an interior portion of the barrel 102, wherein the portion comprises at the barrel distal open face 102d, wherein the plunger seal 104 is in contact with the aqueous composition, thereby sealing and preventing leakage thereof through the barrel distal open face 102d.

It is to be understood that “sealingly movable” as used herein refers to the passage of the plunger seal and plunger head through the barrel, thereby pushing the aqueous composition therethrough proximally, while maintaining sealing. The action is parallel to the passage of a conventional syringe plunger with a seal at its tip, through a syringe barrel.

According to some embodiments, the plunger seal 104 is made of a polymeric material. According to some embodiments, the plunger seal 104 is made of rubber.

According to some embodiments, the plunger actuator 212 is configured to dislocate the plunger head 216 in the proximal direction. According to some embodiments, the plunger actuator 212 is configured to dislocate the plunger head 216 in the proximal direction from a first position to a second position. According to some embodiments, upon said dislocation the plunger head 216 is pressing against and forcing the plunger seal 104 in the proximal direction through the interior portion of the barrel 102. According to some embodiments, upon said dislocation the plunger head 216 is pressing against and forcing the plunger seal 104 in the proximal direction through the interior portion of the barrel 102 thereby forcing at least a portion of the aqueous composition from the barrel 102 to the concentrating module 110. According to some embodiments, upon said dislocation the plunger head 216 is pressing against and forcing the plunger seal 104 in the proximal direction through the interior portion of the barrel 102 thereby forcing at least a portion of the aqueous composition from the barrel 102 to the concentrating module 110 and pushing excess water through the opening 135 to the drain chamber 130.

According to some embodiments, the plunger actuator 212 is configured to dislocate the plunger head 216 in the proximal direction from a first position to a second position, wherein upon said dislocation the plunger head 216 is pressing against and forcing the plunger seal 104 in the proximal direction through the interior portion of the barrel 102, thereby forcing at least a portion of the aqueous composition from the barrel 102 to the concentrating module 110.

According to some embodiments, the means of transferring at least a portion of the aqueous composition from the barrel 102 to the concentrating module 110 comprises a liquid pump. According to some embodiments, the liquid pump is configured to apply pressure on the aqueous composition in the proximal direction, thereby forcing the aqueous composition from the barrel 102 into the concentrating module 110.

It is to be understood that the internal portion of the barrel 102 is a portion of its barrel internal cavity 103 as defined above.

According to some embodiments, the actuator further comprises a controller 220. According to some embodiments, the controller 220 comprises a processing unit. According to some embodiments, the controller 220 is a printed circuit board.

According to some embodiments, the controller 220 is configured to control the operation of the means of transferring at least a portion of the aqueous composition from the barrel 102 to the concentrating module 110. According to some embodiments, the controller 220 is configured to control the operation of the liquid pump. According to some embodiments, the controller 220 is configured to control the operation of the plunger assembly 210.

According to some embodiments, the actuator further comprises a plunger assembly driver 240. According to some embodiments, the controller 220 is electrically connected to the plunger assembly 210 through an electric plunger assembly driver 240. According to some embodiments, the controller 220 is electrically connected to the means of transferring at least a portion of the aqueous composition from the barrel 102 to the concentrating module 110, through an electric driver. According to some embodiments, the controller 220 is electrically connected to the liquid pump through an electric driver.

According to some embodiments, the plunger assembly driver 240 is selected from the group consisting of an electric switch and a transistor. According to some embodiments, electric plunger assembly driver 240 is configured to control the wattage and/or current provided to the plunger assembly 210 from the controller 220. According to some embodiments, electric plunger assembly driver 240 is configured to control the wattage and/or current provided to the to the plunger actuator 212 from the controller 220. According to some embodiments, the electric driver is configured to control the wattage and/or current provided to the liquid pump from the controller 220. As seen in FIGS. 2-8, controller 220 is electrically wired to plunger assembly driver 240, which in its turn is electrically connected to plunger actuator 212 of plunger assembly 210 through an electric wire.

According to some embodiments, the controller 220 is configured to control the operation of the evaporative heater 120.

According to some embodiments, the actuator further comprises an evaporative heater driver 230. According to some embodiments, the controller 220 is electrically connected to the evaporative heater 120 plunger assembly 210 through an evaporative heater driver 230.

According to some embodiments, the evaporative heater driver 230 is selected from the group consisting of an electric switch and a transistor. According to some embodiments, evaporative heater driver 230 is configured to control the wattage and/or current provided to the evaporative heater 120 from the controller 220. As seen in FIGS. 2-8, controller 220 is electrically wired to plunger assembly driver 240, which in its turn is electrically connected to plunger actuator 212 of plunger assembly 210 through an electric wire extending from the actuator 250 to the cartridge 100 through electric contacts (not numbered) incorporated at the aerosol generating device cartridge distal end 100d and matching electric contacts positioned at the actuator proximal face 250p.

According to some embodiments, the actuator 250 has an actuator distal end 250d and an actuator proximal face 250p, wherein the actuator proximal face 250p is connectable to the aerosol generating device cartridge distal end 100d.

According to some embodiments, upon a sufficient amount of the aqueous composition being pushed by the means of transferring from the barrel 102 to the concentrating module 110, the at least one cannabinoid and/or nicotine undergoes stronger retention to the packing material than the water, thereby a dilute cannabinoid and/or nicotine composition is flowing to the drain chamber 130 through the opening 135 and a concentrated cannabinoid and/or nicotine composition is retained within the concentrating module 110; wherein the concentration of the at least one vaporizable compound in the aqueous composition contained in the barrel 102 is higher than the concentration of the at least one vaporizable compound in the dilute cannabinoid and/or nicotine composition and lower than the concentration of the at least one cannabinoid and/or nicotine in the concentrated cannabinoid and/or nicotine composition.

According to some embodiments, the actuator 250 further comprises a power source compartment configured to accommodate a rechargeable battery (not shown). According to some embodiments, the rechargeable battery is configured to provide electric current to the controller 220. According to some embodiments, the rechargeable battery is configured to provide electric current to the evaporation heater 120 through the controller 220 and the evaporative heater driver 230. According to some embodiments, the rechargeable battery is configured to provide electric current to the plunger actuator 212 through the controller 220 and the plunger assembly driver 240. According to some embodiments, battery 194 may be a rechargeable or disposable battery. Specifically, battery 194 may be a relatively strong power source, since, as detailed herein, evaporation heater 120 is configured to generate high electrical wattage. According to some embodiments, the battery is a Lipo battery. According to some embodiments, the battery has voltage of about 3.7V. According to some embodiments, the battery is a battery having voltage of about 3.7V. According to some embodiments, the battery has maximum discharge current of about 40A. According to some embodiments, the battery has capacity of about 1400 mAh (milli-Ampere-hour). According to some embodiments, the battery has C-rating value of about 30. According to some embodiments, the power source compartment comprises the battery.

Method of Producing an Aqueous Cannabinoid and/or Nicotine Aerosol

According to some embodiments, there is provided a method of producing an aqueous cannabinoid aerosol, the method comprising:
(a) providing aqueous composition comprising at least one cannabinoid and/or nicotine;
(b) providing a cartridge comprising a concentrating module and a at least one evaporative heater, wherein the concentrating module having a concentrating module distal open face and a concentrating module proximal open face, wherein the evaporative heater is located proximally to the concentrating module proximal open face;
(c) passing the aqueous composition through the concentrating module, from the concentrating module distal open face towards the evaporative heater, thereby concentrating the aqueous composition; and
(d) operating the at least one evaporative heater, thereby producing an aqueous aerosol.

According to some embodiments, the concentrating module is surrounded by at least one wall and is containing a packing material. According to some embodiments, the packing material has higher affinity to the at least one cannabinoid and/or nicotine than its affinity to water, wherein passing the aqueous composition through the concentrating module in step (c) entails concentrating the aqueous composition. According to some embodiments, the packing material is a chromatographic packing material, wherein passing the aqueous composition through the concentrating module in step (c) entails concentrating the aqueous composition. According to some embodiments, the packing material is any one of the packing material disclosed herein.

According to some embodiments, the concentrating module is surrounded by at least one wall and is containing a packing material, wherein the packing material has higher affinity to the at least one cannabinoid and/or nicotine than its affinity to water, wherein passing the aqueous composition through the concentrating module in step (c) entails concentrating the aqueous composition.

According to some embodiments, the cartridge is the aerosol generating device cartridge 100 described herein.

The aerosol formed by the method of the present invention is shown in FIG. 8 as aerosol 260.

According to some embodiments, step (c) comprises applying liquid pressure of the aqueous composition in the proximal direction.

According to some embodiments, the concentrating the aqueous composition in step (c) comprises separating the aqueous composition into concentrated composition and a dilute composition. According to some embodiments, upon said separation, the concentrated composition resides within the concentrating module. According to some embodiments, upon said separation, the dilute composition resides out of the concentrating module. According to some embodiments, upon said separation, the dilute composition resides out of the barrel. According to some embodiments, upon said separation, the dilute composition resides within the drain chamber.

According to some embodiments, the concentration of the at least one cannabinoid and/or nicotine in the aqueous composition is higher than the concentration of the at least one cannabinoid and/or nicotine in the dilute composition and lower than the concentration of the at least one cannabinoid and/or nicotine in the concentrated composition. According to some embodiments, the concentration of the at least one vaporizable compound in the aqueous cannabinoid composition prior to the separation is higher than the concentration of the at least one vaporizable compound in the dilute composition and lower than the concentration of the at least one vaporizable compound in the concentrated composition.

According to some embodiments, step (c) further comprises flowing the dilute aqueous composition into the drain chamber.

According to some embodiments, the concentration of the at least one cannabinoid and/or nicotine in the aqueous composition is in the range of 1% to 10% w/w. According to some embodiments, concentrating the aqueous composition in step (c) entails at least doubling said concentration. According to some embodiments, concentrating the aqueous composition in step (c) entails at least tripling said concentration. According to some embodiments, concentrating the aqueous composition in step (c) entails at least quadrupling said concentration.

According to some embodiments, the concentration of the at least one vaporizable compound in the concentrated aqueous composition of step (c) is in the range of 10% to 60% w/w. According to some embodiments, the concentration of the at least one vaporizable compound in the concentrated aqueous composition of step (c) is at least 10% w/w. According to some embodiments, the concentration of the at least one vaporizable compound in the concentrated aqueous composition of step (c) is at least 15% w/w. According to some embodiments, the concentration of the at least one vaporizable compound in the concentrated aqueous composition of step (c) is at least 20% w/w. According to some embodiments, the concentration of the at least one vaporizable compound in the concentrated aqueous composition of step (c) is at least 25% w/w. According to some embodiments, the concentration of the at least one vaporizable compound in the concentrated aqueous composition of step (c) is at least 30% w/w. According to some embodiments, the concentration of the at least one vaporizable compound in the concentrated aqueous composition of step (c) is at least 40% w/w.

According to some embodiments, there is provided an aqueous vaporizable compound aerosol produced by the method of the present invention. According to some embodiments, there is provided an aqueous cannabinoid and/or nicotine aerosol 260 produced by the method of the present invention.

According to some embodiments, the aqueous aerosol 260 has droplets having a mass median aerodynamic diameter (MMAD) in the range of 0.2-2 microns.

According to some embodiments, the aqueous aerosol 260 comprises droplets having a mass median aerodynamic diameter (MMAD) of at most 5 microns. According to some embodiments, the aerosol 260 comprises droplets having a mass median aerodynamic diameter (MMAD) of at most 4 microns. According to some embodiments, the aerosol 260 comprises droplets having a mass median aerodynamic diameter (MMAD) of at most 3 microns. According to some embodiments, the aerosol 260 comprises droplets having a mass median aerodynamic diameter (MMAD) of at most 2 microns. According to some embodiments, the aerosol 260 droplets having a mass median aerodynamic diameter (MMAD) of at most 1 micron. According to some embodiments, the aerosol 260 comprises droplets having a mass median aerodynamic diameter (MMAD) of at most 0.8 microns. According to some embodiments, the aerosol 260 comprises droplets having a mass median aerodynamic diameter (MMAD) of at most 0.6 microns. According to some embodiments, the aerosol 260 comprises droplets having a mass median aerodynamic diameter (MMAD) of at most 0.5 microns.

It was surprisingly found that aerosolization of a formulation as disclosed herein, results in droplets having a mass median aerodynamic diameter (MMAD) sufficiently small so as to reach the lungs, rather than precipitate on their way thereto. The small droplets reaching the lungs enable efficient respiratory delivery of the cannabinoid(s) and/or nicotine. This is an overall advantage as maximizing the delivery of cannabinoid(s) and/or nicotine to the lungs, while minimizing its deposition in the mouth and throat are considered highly beneficial.

The terms ‘droplet size’ and ‘mass median aerodynamic diameter’, also known as MMAD, as used herein are interchangeable. MMAD is commonly considered as the median particle diameter by mass. MMAD may be evaluated by plotting droplet size vs. the cumulative mass fraction (%) in the aerosol. MMAD may then be determined according to the interpolated droplet size corresponding to the point, where the cumulative mass fraction is 50%. This points represent the estimated values of particle sizes, above which the droplets are responsible to half to masses and below which the droplets are responsible to the other halves, in each solution.

According to some embodiments, the aerosol 260 comprises droplets having a Geometric Standard Diameter (GSD) within the range of about 0.2-7 micron. According to some embodiments, the aerosol comprises droplets having a GSD within the range of about 0.2-5 micron.

Operation of the Device

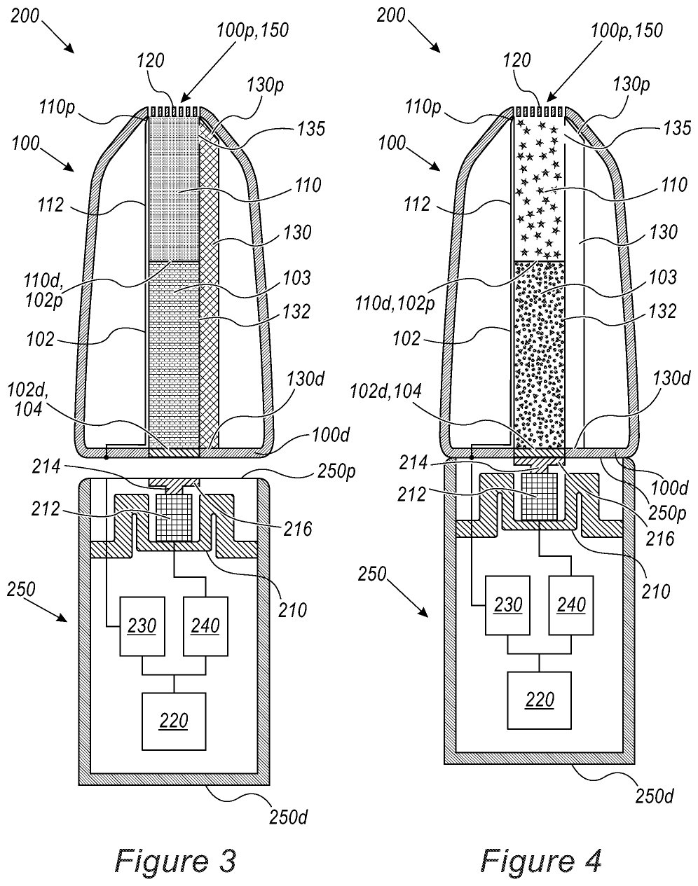

Reference in now made to FIGS. 2 and 3.

FIG. 2 constitutes a schematic cross sectional view of the aerosol generating device 200 comprising the cartridge 100 and the actuator 250, when connected, according to some embodiments. FIG. 3 constitutes a schematic cross sectional view of the aerosol generating device 200 comprising the cartridge 100 and the actuator 250, when separated, according to some embodiments.

Various embodiments directed to the elements and specification of aerosol generating device cartridge 100 are detailed above.

Specifically, according to some embodiments, the actuator 250 is reversibly connectable to the aerosol generating device cartridge 100 at the aerosol generating device cartridge distal end 100d.

FIG. 4 constitutes a schematic cross sectional view of the aerosol generating device 200 comprising the aerosol generating device cartridge 100 and the actuator 250, at stage A of initialization, according to some embodiments.

The cartridge of FIGS. 4-8 contains an aqueous vaporizable compound compositions comprising cannabinoids and/or nicotine (triangles represent either nicotine and/or cannabinoids) and water (circles) and a concentrating module stationary comprising packing material (stars).

It is to be understood that the initialization stage (stage A) of aerosol generating device cartridge 100 and aerosol generating device 200 is the original stage, in which aerosol generating device cartridge 100 is typically provided (i.e. it is purchased that way). Actuator 250 of aerosol generating device 200, on the other hand is typically durable and is intended, according to some embodiments, to be purchased once until requiring maintenance, while replacing plurality of cartridges 100, to be installed therein. Therefore, aerosol generating device cartridge 100 is typically provided with barrel 102 full with an aqueous composition, which may be an aqueous cannabinoids and/or nicotine composition (wherein the triangles represent cannabinoids and/or nicotine) and/or an aqueous nicotine composition (wherein the triangles represent nicotine molecules).

According to some embodiments, concentrating module 110 is provided with an aqueous composition. According to some embodiments, concentrating module 110 is provided with the same aqueous composition, which is present within the barrel 102.

As shown in FIG. 4, the majority of molecules within the barrel 102 are of the water (circles) and the minor portion is of the active compound (e.g., cannabinoid(s) and/or nicotine; triangles). Specifically, the concentration of cannabinoids in water is limited by their aqueous solubility, which prevents higher concentrations, whereas highly concentrated starting nicotine compositions are regulatorically restricted.

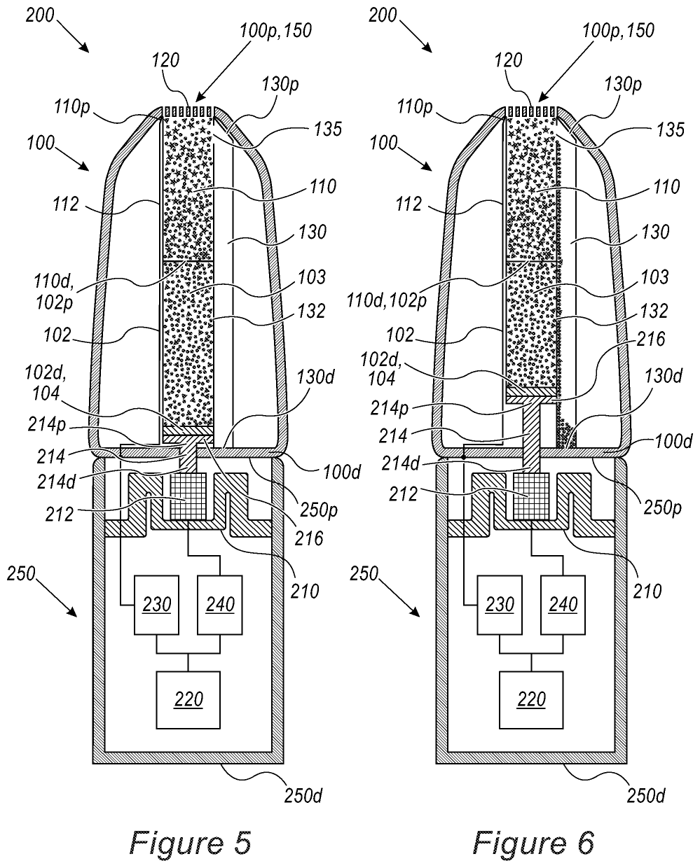

Reference is now made FIG. 5. FIG. 5 constitutes a schematic cross sectional view of aerosol generating device 200 comprising the aerosol generating device cartridge 100 and the actuator 250, at stage B of concentration, according to some embodiments.

As shown in FIG. 5, compared to stage A of FIG. 4, in stage B, the controller 220 is operating plunger assembly 210 by transferring electric current thereto through plunger assembly driver 240, according to some embodiments. The current passed to plunger actuator 212, as detailed herein, according to some embodiments, results in plunger actuator 212 dislocating plunger head 216 in the direction of concentrating module 110, through pushing rod 214. According to some embodiments, this dislocation moves plunger seal 104 in the same direction and forces a portion of the aqueous composition into the concentrating module 110.

When inside the concentrating module 110, the concentration of the active material (cannabinoid(s) and/or nicotine; triangles) is elevated, so that it is higher within the concentrating module 110 than it is within the barrel 102. Specifically, one can notice that the triangle-to-circle ratio is higher in the concentrating module 110 than it is within the barrel 102. More specifically, the packing material, as described in various embodiments herein has relatively high affinity to the active compounds and relatively low affinity to water, so as can be appreciated from FIG. 5, each star (packing material) attracts triangles (active compounds), which are adjacent thereto.

Reference is now made FIG. 6. FIG. 6 constitutes a schematic cross sectional view of aerosol generating device 200 comprising the aerosol generating device cartridge 100 and the actuator 250, at stage C of concentration and drain, according to some embodiments.

Specifically, as in stage B of FIG. 5, in stage C of FIG. 6, the controller 220 is operating plunger assembly 210 by transferring electric current thereto through plunger assembly driver 240; the current passed to plunger actuator 212 results in plunger actuator 212 dislocating plunger head 216 in the direction of concentrating module 110, through pushing rod 214; and, this dislocation moves plunger seal 104 in the same direction and forces an additional portion of the aqueous composition into the concentrating module 110, according to some embodiments.

In stage C of FIG. 6, there is no place left for the aqueous composition in concentrating module 110, so excess of the composition exits concentrating module 110 through opening 135 into drain chamber 130. As detailed herein, the packing material within concentrating module 110 attracts molecules of the active compound. As a result, such compounds, are significantly retained within the drain chamber 130, according to some embodiments, so that the excess evacuated into the drain chamber 130 is dilute in comparison with both the starting aqueous composition within barrel 102, and with the concentrated composition within concentrating module 110. Specifically, one can appreciate from the Figure that the triangle-to-circle ratio is higher in the concentrating module 110 and barrel 102 than it is within the drain chamber 130.

Reference is now made FIG. 7. FIG. 7 constitutes a schematic cross sectional view of aerosol generating device 200 comprising the aerosol generating device cartridge 100 and the actuator 250, at stage D of concentration and drain, according to some embodiments.

Specifically, the same process described above for stage C of concentration and drain in FIG. 6, similarly apply to stage D of concentration and drain in FIG. 7, according to some embodiments. After this additional stage, the concentration of the active material within concentrating module 110 raises and more dilute aqueous composition gathers within drain chamber 130.

Reference is now made FIG. 8. FIG. 8 constitutes a schematic cross sectional view of the aerosol generating device 200 comprising the aerosol generating device cartridge 100 and the actuator 250, at stage E of aerosolization, according to some embodiments.

Specifically, the position of the plunger head 216 is substantially similar in FIG. 7 and FIG. 8, according to some embodiments. The difference is the actuation of the evaporative heater 120.

As shown in FIG. 8, the controller 220 is operating evaporative heater 120 by transferring electric current thereto through evaporative heater driver 230, according to some embodiments. The current passed to evaporative heater 120, as detailed herein, according to some embodiments, results in evaporative heater 120 raising the temperature in the vicinity of the concentrated aqueous composition. As a result, according to some embodiments, the concentrated aqueous composition is vaporized and then turns into an aerosol 260.

When inside the concentrating module 110, the active material (cannabinoid(s) and/or nicotine; triangles) is more concentrated than it is originally within the barrel 102. As the aqueous composition concentration in the vicinity of the evaporative heater 120 is similar to that inside concentrating module 110, the concentration of cannabinoids and/or nicotine in the aerosol 260, is similar to that within concentrating module 110, according to some embodiments. Specifically, one can notice that the triangle-to-circle ratio is similar in the concentrating module 110 and in the aerosol 260.

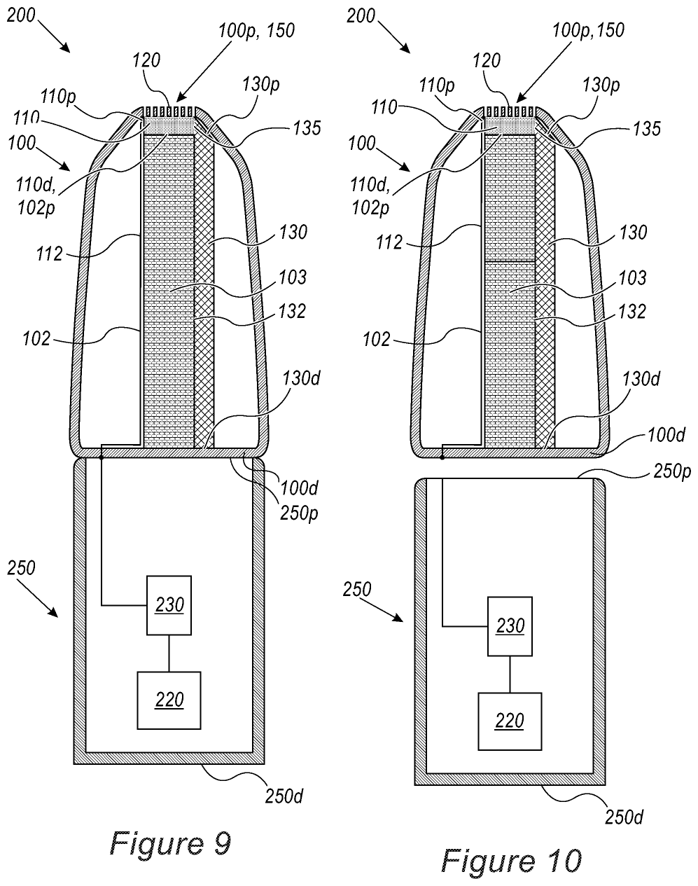

Reference in now made to FIGS. 9 and 10.

FIG. 9 constitutes a schematic cross-sectional view of the aerosol generating device 200 comprising the cartridge 100 and the actuator 250, when connected, according to some embodiments. FIG. 10 constitutes a schematic cross-sectional view of the aerosol generating device 200 comprising the cartridge 100 and the actuator 250, when separated, according to some embodiments.

Various embodiments directed to the elements and specification of aerosol generating device cartridge 100 are detailed above.

Specifically, according to some embodiments, the actuator 250 is reversibly connectable to the aerosol generating device cartridge 100 at the aerosol generating device cartridge distal end 100d.

Differences between the aerosol generating device 200 and the cartridge 100 of FIGS. 9 to 12 and the respective aerosol generating device 200 and the cartridge 100 of FIGS. 2 to 8, stem from the different modes of operation. Specifically, according to some embodiments, the aerosol generating device 200 of FIGS. 2 to 8 operates in an “active” liquid delivery and concentration mode, whereas the aerosol generating device 200 of FIGS. 9 to 12 operates in an “passive” liquid delivery and concentration mode.

More particularly, as described in the description of FIGS. 2 to 8, the aqueous composition inside the cartridge 100 of FIGS. 9 to 12 is similarly flowing from the barrel 102 towards the barrel proximal open face 102p, where it penetrates the concentrating module 110 through the concentrating module proximal open face 110p, according to some embodiments. In the cartridge 100 of FIGS. 9 to 12, however, the barrel distal face is closed, opposed to barrel distal open face 102d in the cartridge 100 of FIGS. 9 to 12. Thus, the cartridge 100 of FIGS. 9 to 12 does not include, according to some embodiments, the plunger seal 104 of FIGS. 2 to 8, according to some embodiments. Also, according to some embodiments, in contrast with the actuator 250 of FIGS. 2 to 8, the actuator 250 of FIGS. 9 to 12 does not include the plunger assembly of FIGS. 2 to 8 (i.e., the plunger assembly 210, the plunger actuator 212, the rod 214 and the plunger head 216). Instead, the cartridge 100 of FIGS. 9 to 12 includes a liquid absorbing element 1032, according to some embodiments. Some of the distinct structural features and elements of the cartridge 100 of FIGS. 9 to 12 will be described below, followed by explanations about the “passive” liquid delivery and concentration mode and approach.

According to some embodiments, the barrel 102 comprises a liquid absorbing element 1032. According to some embodiments, the barrel 102 is housing a liquid absorbing element 1032. According to some embodiments, the liquid absorbing element 1032 resides within the barrel internal cavity 103. According to some embodiments, the liquid absorbing element 1032 is extending through the barrel 102. According to some embodiments, the liquid absorbing element 1032 is elongated. According to some embodiments, the liquid absorbing element 1032 is in fluid communication with the concentrating module 110. According to some embodiments, the liquid absorbing element 1032 is in fluid communication with the concentrating module 110 through concentrating module distal open face 110d. According to some embodiments, the liquid absorbing element 1032 is in contact with the concentrating module 110. According to some embodiments, the liquid absorbing element 1032 is in contact with the concentrating module distal open face 110d. According to some embodiments, the barrel 102 comprises a liquid absorbing element 1032, which is in contact with the concentrating module distal open face 110d. According to some embodiments, the liquid absorbing element 1032 is attached to the barrel 102. According to some embodiments, the liquid absorbing element 1032 is a stationary liquid absorbing element.

According to some embodiments, the liquid absorbing element 1032 comprises a sponge, a wick or both. According to some embodiments, the liquid absorbing element 1032 is a sponge, a wick or both. According to some embodiments, the liquid absorbing element 1032 is selected from the group consisting of: a sponge, a wick and a combination thereof. According to some embodiments, the liquid absorbing element 1032 is a sponge. According to some embodiments, the liquid absorbing element 1032 is a wick. According to some embodiments, the liquid absorbing element 1032 comprises a sponge. According to some embodiments, the liquid absorbing element 1032 comprises a wick.

According to some embodiments, the barrel 102 further comprises at least one container 1031. According to some embodiments, the container 1031 is in fluid communication with the liquid absorbing element 1032. According to some embodiments, the container 1031 is in contact with the liquid absorbing element 1032. According to some embodiments, the container 1031 contains part of the aqueous composition. According to some embodiments, another part of the aqueous composition is absorbed within the liquid absorbing element 1032. According to some embodiments, the barrel further comprises at least one container 1031, which is in fluid communication with the liquid absorbing element 1032 and contains part of the aqueous composition, wherein another part of the aqueous composition is absorbed within the liquid absorbing element 1032.

The cross-section of the liquid absorbing element 1032 in FIGS. 10-12 is portrayed as an elongated T shape, which is a non-limiting example of an optional structure thereof, according to some embodiments. Also, in these figures, a single container 1031 is surrounding the liquid absorbing element 1032 along the majority of the barrel 102, according to some embodiments. Thus, according to some embodiments, the perimeter of proximal end of the liquid absorbing element 1032 is substantially fitting the perimeter of the concentrating module distal open face 110d.

According to some embodiments, the controller 220 of the actuator 250 described in FIGS. 9 to 12 is configured to control the operation of the evaporative heater 120, similarly to the mode described when referring to FIGS. 2 to 8.

Specifically, these certain differences in structure between the aerosol generating device cartridges 100 and aerosol generating devices 200 of FIGS. 2 to 8 and the corresponding devices of FIGS. 9 to 12, stems from the active mode of operation of the former versus the passive mode of operation of the latter. Whereas the active mode is elaborated above when referring to FIGS. 2 to 8, the devices of FIGS. 9 to 12 are based on capillary motion of aqueous compositions within porous absorbing materials, such as sponge, wick and the like. Through the operation of the aerosol generating devices 200 and cartridge 100 of FIGS. 9 to 12, the aqueous composition within the at least one container 1031 is being absorbed by the liquid absorbing element 1032 until it is substantially saturated, such that the aqueous composition commences to flow towards the concentrating module 110. The flow of the aqueous composition within the concentrating module 110, the variation in concentration and evaporation using evaporative heater 120 to form aerosol 260 are all described and similar to the corresponding step described with relation to FIGS. 2-8. After the aerosolization occurs, empty space is forming instead of the vacated aqueous composition, followed by additional flow of another portion of the aqueous composition from the liquid absorbing element 1032 to the concentrating module 110. Afterwards, a similar portion of the aqueous composition from the at least one container 1031 is being absorbed by the liquid absorbing element 1032 until the liquid absorbing element 1032 substantially saturates again, according to some embodiments. This action may repeat until the aqueous composition within the at least one container 1031 is consumed.

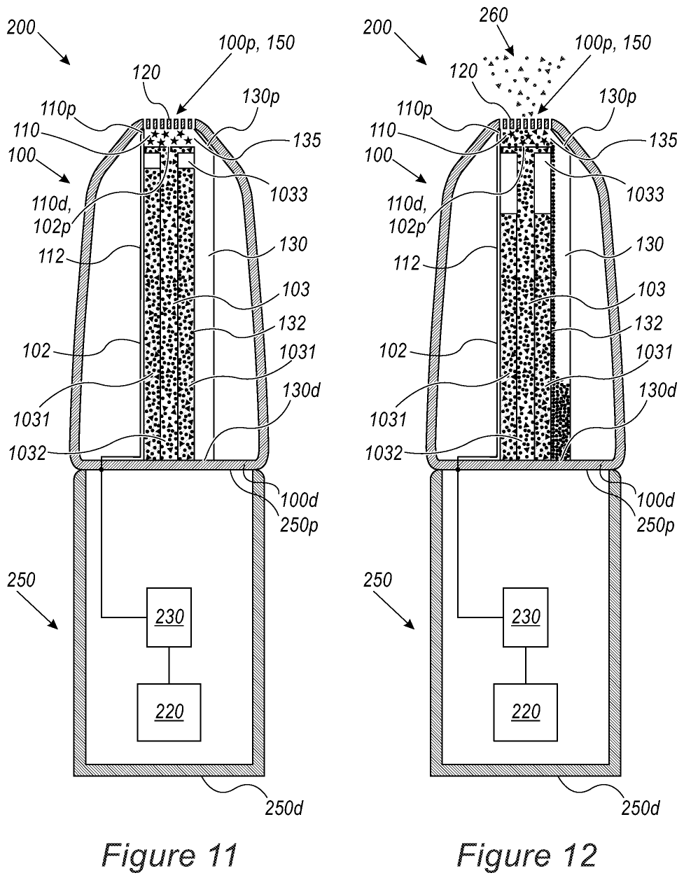

FIG. 11 constitutes a schematic cross-sectional view of the aerosol generating device 200 comprising the aerosol generating device cartridge 100 and the actuator 250, at stage A of initialization, according to some embodiments.

The cartridge 100 of FIGS. 11-12 contains an aqueous vaporizable compound compositions comprising cannabinoids and/or nicotine (triangles represent either nicotine and/or cannabinoids) and water (circles) and a concentrating module stationary comprising packing material (stars).

It is to be understood that the initialization stage (stage A) of aerosol generating device cartridge 100 and aerosol generating device 200 is the original stage, in which aerosol generating device cartridge 100 is typically provided (i.e., it is purchased that way). Actuator 250 of aerosol generating device 200, on the other hand is typically durable and is intended, according to some embodiments, to be purchased once until requiring maintenance, while replacing plurality of cartridges 100, to be installed therein. Therefore, aerosol generating device cartridge 100 is typically provided with at least one container 1031 substantially full with an aqueous composition, which may be an aqueous cannabinoid and/or nicotine composition (wherein the triangles represent cannabinoids and/or nicotine) and/or an aqueous nicotine composition (wherein the triangles represent nicotine molecules).

According to some embodiments, concentrating module 110 is provided with an aqueous composition. According to some embodiments, concentrating module 110 is provided with the same aqueous composition, which is present within the barrel 102.

As shown in FIG. 11, the majority of molecules within the barrel 102 (including the at least one container 1031 and the liquid absorbing element 1032) are of the water (circles) and the minor portion is of the active compound (e.g., cannabinoid(s) and/or nicotine; triangles).

Stages B-D of concentration in the aerosol generating device 200 and cartridge 100 of FIGS. 9-12 are not shown in the figures and can be appreciated by the skilled in the art based on the present text and figures.

With reference to the device 200 and cartridge 100 of FIGS. 9-12, compared to stage A, in stage B, a portion of the aqueous composition is passing from liquid absorbing element 1032 into the concentrating module 110. Consequently, a portion of the aqueous composition having similar volume passes from the at least one container 1031 towards the liquid absorbing element 1032. As a result, the at least one container 1031 begins to empty, such that a void 1033 is developing/increasing within the at least one container 1031.

When inside the concentrating module 110, the concentration of the active material (cannabinoid(s) and/or nicotine; triangles) is elevated, so that it is higher within the concentrating module 110 than it is within the barrel 102 (i.e., within the at least one container 1031 and absorbed in the liquid absorbing element 1032).

Specifically, as in stage B, in stage C, a portion of the aqueous composition is passing from liquid absorbing element 1032 into the concentrating module 110. Consequently, a portion of the aqueous composition having similar volume passes from the at least one container 1031 towards the liquid absorbing element 1032. As a result, the at least one container 1031 begins to empty, such that a void 1033 is increasing within the at least one container 1031.

In stage C there is no place left for the aqueous composition in concentrating module 110, so excess of the composition exits concentrating module 110 through opening 135 into drain chamber 130. As detailed herein, the packing material within concentrating module 110 attracts molecules of the active compound. As a result, such compounds, are significantly retained within the drain chamber 130, according to some embodiments, so that the excess evacuated into the drain chamber 130 is dilute in comparison with both the starting aqueous composition within barrel 102 (including both the at least one container 1031 and the liquid absorbing element 1032), and with the concentrated composition within concentrating module 110.

The same process described above for stage C of concentration and drain, similarly apply to stage D of concentration and drain, according to some embodiments. After this additional stage, the concentration of the active material within concentrating module 110 raises, more dilute aqueous composition gathers within drain chamber 130 and the void 1033 within at least one container 1031 increases.

Reference is now made FIG. 12. FIG. 21 constitutes a schematic cross-sectional view of the aerosol generating device 200 comprising the aerosol generating device cartridge 100 and the actuator 250, at stage E of aerosolization, according to some embodiments.

As shown in FIG. 12, the controller 220 is operating evaporative heater 120 by transferring electric current thereto through evaporative heater driver 230, according to some embodiments. The current passed to evaporative heater 120, as detailed herein, according to some embodiments, results in evaporative heater 120 raising the temperature in the vicinity of the concentrated aqueous composition. As a result, according to some embodiments, the concentrated aqueous composition is vaporized and then turns into an aerosol 260.

When inside the concentrating module 110, the active material (cannabinoid(s) and/or nicotine; triangles) is more concentrated than it is originally within the at least one container 1031. As the aqueous composition concentration in the vicinity of the evaporative heater 120 is similar to that inside concentrating module 110, the concentration of cannabinoids and/or nicotine in the aerosol 260, is similar to that within concentrating module 110, according to some embodiments. Specifically, one can notice that the triangle-to-circle ratio is similar in the concentrating module 110 and in the aerosol 260.

It is understood that aspect and embodiments described herein include “consisting” and/or “consisting essentially of” aspects and embodiments. As used herein, the singular form “a”, “an”, and “the” includes plural references unless indicated otherwise.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An aerosol generating device cartridge having a proximal end and a distal end, wherein the cartridge comprises:
a barrel having a proximal open face and a distal face, wherein the distal face is facing and located proximally to the distal cartridge end, wherein the barrel comprises an aqueous composition comprising at least one vaporizable compound selected from: a cannabinoid, nicotine, or both;
a concentrating module having a distal open face and a proximal open face, wherein the concentrating module distal open face is in contact with the barrel, wherein the concentrating module is surrounded by at least one wall and is containing a packing material, wherein the packing material has higher affinity to the at least one vaporizable compound than its affinity to water;
an outlet at the cartridge proximal end; and
at least one evaporative heater configured to generate heat and to evaporate water from a surface thereof, wherein the evaporative heater is located proximally to the concentrating module proximal open face.

2. The cartridge of claim 1, further comprising a drain chamber, wherein the at least one concentrating module wall comprises an opening, wherein the opening is allowing fluid communication between the drain chamber and the concentrating module.

3. The cartridge of claim 2, wherein the drain chamber is a substantially closed chamber, having at least one wall shared with the at least one concentrating module wall, wherein said shared wall comprises said opening, to allow the fluid communication.

4. The cartridge of claim 2, wherein upon a sufficient amount of the aqueous composition being transferred from the barrel to the concentrating module, the at least one vaporizable compound undergoes stronger retention to the packing material than the water, thereby a dilute vaporizable compound composition is flowing to the drain chamber through the opening and a concentrated vaporizable compound composition is retained within the concentrating module; wherein the concentration of the vaporizable compound in the aqueous composition contained in the barrel is higher than the concentration of the vaporizable compound in the dilute composition and lower than the concentration of the vaporizable compound in the concentrated composition.

5. (canceled)

6. (canceled)

7. The cartridge of claim 1, wherein the vaporizable compound is a cannabinoid, wherein the packing material has higher affinity to the cannabinoid than its affinity to water.

8. The cartridge of claim 1, wherein the vaporizable compound is nicotine, wherein the packing material has higher affinity to nicotine than its affinity to water.

9. (canceled)

10. The cartridge of claim 1, wherein the packing material is selected from the group consisting of: activated carbon, an ion exchange chromatography stationary phase packing material, a reversed-phase chromatography stationary phase packing material, a size exclusion chromatography stationary phase packing material and a combination thereof.

11. The cartridge of claim 1, wherein the packing material phase packing material comprises silica, alumina, zirconia, titania, a cross linked polymer, a derivative or combination thereof.

12. (canceled)

13. (canceled)

14. (canceled)

15. (canceled)

16. (canceled)

17. (canceled)

18. (canceled)

19. (canceled)

20. The cartridge of claim 7, wherein the cannabinoid is selected from the group consisting of cannabidiol, tetrahydrocannabinol, cannabidiolic acid, tetrahydrocannabinolic acid, salts thereof and combinations thereof.

21. (canceled)

22. The cartridge of claim 7, wherein the cannabinoid acid is selected from the group consisting of tetrahydrocannabinolic acid, cannabidiolic acid and salts thereof.

23. The cartridge of claim 7, wherein the aqueous cannabinoid composition has a pH higher than 9.

24. (canceled)

25. The cartridge of any claim 1, wherein the evaporative heater is configured to generate heat and to evaporate the aqueous composition from the surface thereof to form an aqueous aerosol comprising the at least one vaporizable compound, wherein the aqueous aerosol has a pH in the range of 5.5 to 7.5.

26. (canceled)

27. (canceled)

28. The cartridge of any claim 4, wherein the concentration of the vaporizable compound in the aqueous composition contained in the barrel is in the range of 1% to 5% w/w and the concentration of the vaporizable compound in the concentrated composition in the range of 10% to 90% w/w.

29. (canceled)

30. (canceled)

31. The cartridge of claim 1, wherein the evaporative heater is at least partially permeable to the aqueous formulation or wherein the cartridge comprises a plurality of evaporative heaters, wherein upon application of pressure of a fluid against the heater or the heaters, the fluid can pass therethrough.

32. (canceled)

33. (canceled)

34. (canceled)

35. (canceled)

36. (canceled)

37. (canceled)

38. The cartridge of claim 1, wherein the barrel comprises a liquid absorbing element, which is in contact with the concentrating module distal open face, wherein the barrel further comprises at least one container, which is in fluid communication with the liquid absorbing element and contains part of the aqueous composition, wherein another part of the aqueous composition is absorbed within the liquid absorbing element.

39. (canceled)

40. (canceled)

41. An aerosol generating device comprising the cartridge of claim 1 and an actuator, wherein the actuator is reversibly connectable to the cartridge at the cartridge distal end, wherein the aerosol generating device further comprises means of transferring at least a portion of the aqueous composition from the barrel to the concentrating module.

42. The aerosol generating device of claim 41, wherein the means of transferring at least a portion of the aqueous composition from the barrel to the concentrating module comprises a plunger assembly.

43. The aerosol generating device of claim 42, wherein the plunger assembly comprises a plunger actuator, a rod and a plunger head, wherein the rod has a proximal end and a distal end, wherein the distal end is connected to the solenoid actuator, and the proximal end is connected to the plunger head, wherein the plunger actuator is configured to dislocate the plunger head in the proximal direction from a first position to a second position, thereby to force at least a portion of the aqueous composition from the barrel to the concentrating module.

44. (canceled)

45. The aerosol generating device of claim 44, further comprising a plunger seal sealingly movable through at least an interior portion of the barrel, wherein the portion comprises at the distal barrel face, wherein the plunger seal is in contact with the aqueous composition, thereby sealing and preventing leakage thereof through the distal barrel face, wherein the plunger actuator is configured to dislocate the plunger head in the proximal direction from a first position to a second position, wherein upon said dislocation the plunger head is pressing against and forcing the plunger seal in the proximal direction through the interior portion of the barrel, thereby forcing at least a portion of the aqueous composition from the barrel to the concentrating module.

46. A method of producing an aqueous aerosol, the method comprising:
(a) providing aqueous composition comprising at least one vaporizable compound selected from: a cannabinoid, nicotine, or both;
(b) providing a cartridge comprising a concentrating module and a at least one evaporative heater, wherein the concentrating module having a distal open face and a proximal open face, wherein the evaporative heater is located proximally to the concentrating module proximal open face;
(c) passing the aqueous composition through the concentrating module, from the distal concentrating module open face towards the evaporative heater, thereby concentrating the aqueous composition; and
(d) operating the at least one evaporative heater, thereby producing an aqueous aerosol.

47. (canceled)

48. (canceled)

49. (canceled)
